(19) United States
(12) Patent Application Publication
(10) Pub. No.: US 2020/0075152 A1
(43) Pub. Date: Mar. 5, 2020

(54) Fitness nutrition tracking and recommendation service

(71) Applicant: Sergio Radovcic, Phoenix, AZ (US)

(72) Inventor: Sergio Radovcic, Phoenix, AZ (US)

(21) Appl. No.: 16/122,491

(22) Filed: Sep. 5, 2018

(51) Int. Cl. G16H 20/60; G16H 20/30; A61B 5/00; G06K 19/077

(52) U.S. Cl. CPC G16H 20/60; G06K 19/07762; A61B 5/0022; G16H 20/30

(57) ABSTRACT

An executable network-hosted service model includes at least one server controlled by a service provider entity, the at least one server connected to the network and to at least one data repository, software executed by the server from a non-transitory computer readable medium coupled to or otherwise accessible to the server, the software including at least one set of machine readable instructions to operate a service that takes in activity data recorded by at least one dedicated activity data tracking device controlled by a service beneficiary having a medical condition, processing the recorded data on behalf of the beneficiary, providing the data results for view by the beneficiary along with recommendations to consume available vitamins and nutrient supplements especially formulated for the medical condition of the beneficiary.

### CROSS-REFERENCE TO RELATED DOCUMENTS

None

### BACKGROUND OF THE INVENTION

### 1. Field of the Invention

The present invention is in the field of physical and nutritional fitness and pertains particularly to methods and apparatus for tracking general activity of users and providing recommendations relating to nutritional needs.

### 2. Discussion of the State of the Art

In the field of physical fitness, maintaining physical health and fitness is a concerted passion for people all over the world. Aside from user-operated fitness machines, many consumer products have been developed to help people track their activities and pursue goals related to fitness. These products include mobile and often wearable electronics devices for tracking activity and certain states of a user performing the tracked activity at the time. An activity that may be tracked might be running, walking, swimming, dieting, lifting, cycling, and other activities pursued with a goal of attaining a level of physical fitness for the tracked user. It is estimated that over 2 million users are currently tracking their activities and pursuing some fitness goal.

Modular electronics devices are known to be able to track distance and time, running or walking, monitor heart rates, and blood pressures, sweat rates, breathing rates, and can calculate simple results such as the number of calories burned, electrolyte loss and so on during the activity. Mobile phone applications have been created that enable a user being tracked to upload data to a remote server where the data may be processed, and recommendations may be later sent to the user being tracked. Some of these products include but are not limited to running watchers, mobile phone apps, such as RunKeeper or Strava, or like activity trackers, such as FitEit or Jawbone. Forums are also a popular way of tracking physical fitness and health. These also allow the user to receive detailed feedback and recommendations. More particularly, these products work individually and do not aggregate the data from disparate data sources to produce personalized recommendations.

Currently, many products and mobile applications exist that may provide little or no generalized feedback to a user relative to attaining or improving upon on their health and fitness goals. People have differing needs based on their activities and other events. Without proper data aggregation, feedback may be sparse or even detrimental to the user due to variables that are not tracked or are unaccounted for. The inventor is aware of a tracking and feedback process included herein as a reference US patent application entitled Process for Converting actual Fitness Data into Nutritional Advice U.S. Ser. No. 14/823,837.

The process referenced above describes a method for acquisition of fitness data and outputting personalized user recommendations. The process involves searching for one or more tracking devices being operated by a user, connecting to the device and uploading the fitness data to a mobile phone executing an application. The application on the phone may solicit data from the user through the application such as by prompt and reception of answers to fitness questions, and prompt and reception of answers to general questions about health, diet, and behavior. This data may be aggregated for that user and then processed along with fitness data from tracked activity to determine if any recommendations might be made to the user according to the fitness data, solicited data, and knowledge data provided by one or more nutritional experts. The user may be shown an application results page for example, displaying recommendations including rational for any recommendations made.

There is a desire that nutritional information, especially recommendations or advice be relevant to an individual user as opposed to being general information based upon what may be beneficial to a general group of people. Current fitness tracking and reporting apparatus fails to provide personalized or customized recommendations to an extent that medical conditions such as pregnancy, cancer, diabetes, are typically not considered and temporary hormonal cycles such as mensuration, menopausal, and testosterone type conditions are overlooked as are many drugs including steroids that may be ingested.

Therefore, what is clearly needed is a data tracking analysis and reporting system that combines real time tracked fitness data with solicited data and third-party knowledge data to create and communicate interactive recommendations that are highly personalized for everyone.

### BRIEF SUMMARY OF THE INVENTION

An executable network-hosted service model is provided and includes at least one server controlled by a service provider entity, the at least one server connected to the network and to at least one data repository, software executed by the server from a non-transitory computer readable medium coupled to or otherwise accessible to the server, the software including at least one set of machine readable instructions, the instructions causing the server to perform the tasks (a) receiving over the network from a service beneficiary having a current medical condition, activity data recorded by at least one dedicated activity data tracking device controlled by the service beneficiary, the at least one data tracking device adapted for at least short range wireless communication with at least one other electronics device, (b) identifying the service beneficiary, locating data previously stored about the service beneficiary and general knowledge data stored about the current medical condition of the service beneficiary, and mapping the activity data received in (a) to the located beneficiary data and knowledge data, (c) retrieving available third party held data from one or more other servers connected to the network, the third party data requested having at least some relevancy to the recorded activity data, (d) processing the activity data for calculable data results, the calculation performed in light of the information stored about the service beneficiary including knowledge data, and in light of the third party data retrieved in (c), (e) updating the data stored about the service beneficiary in the at least one data repository with the new data results calculated in (d), (f) requesting through at least one generated recommendation notification sent to the service beneficiary over the network at least one transaction item to be initiated by the service beneficiary and fulfilled by the service provider, and (g) completing at least one transaction item initiated by the service beneficiary in (f), including fulfilling the order of the transaction.

In one embodiment, the at least one dedicated activity tracking device includes a wearable electronic pendant including a sensor tracking at least motion, time of tracked motion, and direction and distance of tracked motion. In one embodiment, the service beneficiary is a woman and the current medical condition is a pregnancy, the service model adapted to determine and address at least the nutritional needs of the service beneficiary through a pregnancy of approximately 280 days and during a post pregnancy period of approximately 720 days. In one embodiment, the data stored about the service beneficiary includes a labs history documenting nutritional intake and baseline health data compiled through repetitive data tracking, result processing, and appendage to the stored data during the service period of pregnancy plus 720 days of early childhood development of the child.

In one embodiment of the invention, the at least one dedicated data tracking device includes a dedicated electronic liquid containment bottle including a sensor for tracking the amount of liquid the service beneficiary drinks over time. In a variation of this embodiment, the service beneficiary employs both the pendant tracking device and the liquid consumption tracking device for a same period of activity tracking.

In one embodiment, the activity tracking device communicates tracked data wirelessly to a smart phone controlled by the service beneficiary, wherein the service beneficiary uses the smart phone to navigate online and transfer the tracked activity data to the at least one server over the network the transfer aided by a thin client smart phone application providing connectivity between the service beneficiary and the service provider. In another embodiment of the invention, the at least one dedicated data tracking device includes a dedicated electronic weigh scale including a bio electric impedance program and apparatus for dividing total weight into sub-categories of total weight including at least lean muscle mass, bone mass, and visceral fat percentage.

In one embodiment, the activity data recorded by the at least one dedicated tracking device in (a) includes at least one date and time-stamped file created by the service beneficiary as a bookmarked moment created sometime during activity tracking by physically manipulating a user interface indicium provided on the activity tracker. In one embodiment, the third party held data retrieved in (c) is weather data including actual and forecast data, location data including global position and sea level-based elevation data.

In one embodiment of the present invention, the service beneficiary receives notification of one or more moments bookmarked by the service beneficiary and may associate one or more media files to each bookmarked moment and wherein the service beneficiary may provide authorization to the service provider to publish the one or more characterized moments to a list of friends and family made available to the service provider by the service beneficiary.

In one embodiment of the present invention, in (f), the order of the transaction is for vitamins, protein supplements, electrolytes, or a combination thereof for one or more of three trimesters of pregnancy or one or a combination thereof for a postnatal period. In one embodiment, in (f), the order of the transaction is for one or a combination of organic diapers (such as bamboo), organic baby wipes, organic lotions, organic powders, and baby foods or formulas.

In a preferred embodiment, the service beneficiary uses the smart phone aided by the thin client application to view logged data results and to receive notifications of service adjustments to logged data, reminders to take vitamins, reminders to weigh in and report weight data, and notifications requesting authorization to make adjustments to purchase orders with more or less product or to initiate a transaction for a new product. In a preferred embodiment, the vitamins and the nutrients recommended for the service beneficiary are strategically mixed for each of the three trimesters of pregnancy and for the postnatal period of the service based on a combination of knowledge solicited or contributed and normalized baseline nutritional data logged for the service beneficiary.

In one embodiment, the wearable electronic pendant further includes a sensor for tracking exposure to sunlight, and micro LED powered readouts displaying percentage of or total steps taken, percentage of or total ounces of liquids consumed, and percentage of or total calories burned. In a variation of this embodiment, the wearable electronic pendant may further be worn as a wrist device, or a belt clipped device by installing the pendant into the wearable forms.

In a preferred embodiment, the general knowledge data stored about the current medical condition of the service beneficiary includes at least knowledge data about nutritional deficiencies that may be caused by the medical condition, knowledge data about metabolic shifts that may occur during the medical condition, and knowledge data about hydration level shifts that may occur during the medical condition. In one embodiment, the network is the Internet network including any connected sub networks.

### BRIEF DESCRIPTION OF THE SEVERAL VIEWS OF THE DRAWINGS

FIG. 1 is an architectural overview of a communications network supporting activity tracking and product fulfillment according to an embodiment of the present invention.

FIG. 2 is a process flow chart depicting steps for beginning tracking and recommendation services for a client.

FIG. 3 is a process flow chart depicting the process of FIG. 2 continued.

FIG. 4A is a top view of the pendant of FIG. 1 connected to a USB charge cradle according to an embodiment of the invention.

FIG. 4B is a top view of a polymer jacket adapted to hang the pendant device about the neck in typical pendant wear position.

FIG. 4C is a top view of a polymer jacket adapted to clip the pendant onto a belt worn by the client.

FIG. 4D is a front perspective view of a flexible wrist band jacket.

FIG. 5 is a block diagram depicting basic electronics components in footprint 500 of the pendant of FIG. 4A.

FIG. 6 is an elevation view of the smart water bottle of FIG. 1.

FIG. 7 is a top view of bottle the of FIG. 6.

FIG. 8 is a block diagram depicting basic electronics components contained in the cap of FIG. 6.

FIG. 9A is a top view of the scale of FIG. 1.

FIG. 9B is a bottom view of the scale of FIG. 1.

FIG. 9C is a block diagram depicting basic circuitry components in space 906 of FIG. 9B.

FIG. 10 is a front elevation view of the smart blender device of FIG. 1.

FIG. 11A is a top view of the blender device with the blender container and blades removed.

FIG. 11B is a block diagram depicting basic electronics components of the blender of FIG. 1.

FIG. 12 is a sequence diagram depicting basic network interaction between the components of the system of the invention.

FIG. 13 is a block diagram depicting basic software layers of the core service application combining results processing and recommendation processing.

FIG. 14 is a block diagram depicting functional connectivity between basic components of the service model of the present invention.

FIG. 15 is a block diagram of the knowledge base repository of FIG. 1.

FIG. 16 is a process flow chart depicting steps for tracking a moment and associating media to the moment according to an embodiment of the present invention.

FIG. 17 is a united modeling language diagram depicting a business and service model representing an abstract business concept model for practicing the invention from a service provider perspective.

FIG. 18 is a block diagram depicting a set of exemplary application screen shots of the client application of FIG. 1.

FIG. 19 is a block diagram depicting a set of exemplary application screen shots of the client application of FIG. 1.

FIG. 20 is a block diagram depicting a set of exemplary application screen shots of the client application of FIG. 1.

FIG. 21 is a block diagram depicting a set of exemplary application screen shots of the client application of FIG. 1.

FIG. 22 is a block diagram depicting a set of exemplary application screen shots of the client application of FIG. 1.

FIG. 23 is a block diagram depicting an artificial intelligence (AI) interaction sequence between a system personal coaching application and a pregnant client.

FIG. 24 is a perspective view of a pregnancy kit package adapted to hold specially mixed vitamins for a client, the vitamins separated in the packaging by trimester that they are mixed for.

### DETAILED DESCRIPTION OF THE INVENTION

In various embodiments described in enabling detail herein, the inventor provides a unique system tracking activity and for recommending nutrients activities and promoting wellbeing of a fitness client having a temporary physical condition such as a pregnancy. The present invention is described using the following examples, which may describe more than one relevant embodiment falling within the scope of the invention. The inventor provides a platform and service for tracking activities of pregnant clients and fulfilling their nutritional needs and the nutritional needs of their babies. The present invention is described in enabling detail using the following examples, which may describe more than one relevant embodiment falling within the scope of the present invention.

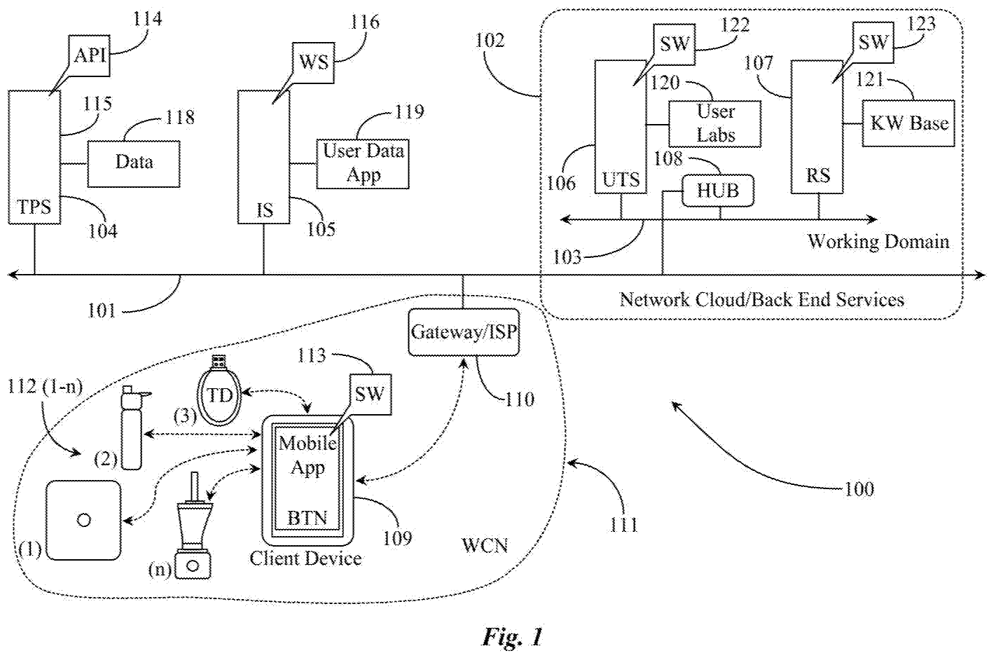

FIG. 1 is an architectural overview of a communications network 100 supporting activity tracking and product fulfillment according to an embodiment of the present invention. Communications network 100 includes the well-known Internet network referenced herein as a network backbone 101. Network backbone 101 represents all the lines, equipment, and access points that make up the Internet network including any connected sub-networks, therefore, there are no geographic limitations on the practice of the present invention. Network backbone 101 may also be referred to in this specification as Internet 101. Backbone 101 may be that of a corporate wide-area-network (WAN), a private WAN, a municipal area network (MAN) without departing from the spirit and scope of the present invention.

Internet 101 is accessible through a gateway 110 hosted by an Internet service provider (ISP). Gateway 110 may bridge a wireless carrier network (WCN) 111 to Internet 101 for connectivity. Internet 101 supports an information server (IS) 105 adapted to serve web pages and websites to users upon user request. Server 105 hosts a website (WS) 116. WS 116 may serve as an access point for clients to join and subscribe to the service of the invention. Server 105 has connection to a data repository 118 adapted to contain data about subscribed clients and data about a client software (SW) application 113 that may be provided for download to clients wishing to join the network.

SW 113 is depicted on a client smart phone 109 that has access to Internet 101, server 105, and website 116, through gateway 110. SW 113 may be downloaded and installed on smart phone 109 by an operating client. In this embodiment, an operating client is a pregnant woman seeking to help herself and her baby to flourish nutritionally during pregnancy and after the baby is born. SW 113 is a dedicated thin client application that provides a vehicle for passing activity data to the tracking and recommendation service for processing and a user interface for ordering nutritional supplements and other products that are specially adapted for pregnant women and their babies.

A client operating smart phone 109 aided by SW 113 may have access to one or more activity trackers also referred to herein as a tracking device (TD) 112 (3) of a group of electronics devices 112 (1-n) that may be available to the client operating smart phone 109. Tracking device 112 (3) is an electronic activity tracker that functions to track movement of the client, like walking, running and other motion-based activities like swimming, exercises, including sleep (intermittent movements in a single location inferring a sleep activity).

TD 112 (3) is in the physical form of a pendant and may be suspended from a tether about the neck of the client as would a pendant jewelry piece. TD 112 (3) may be referred to herein as pendant 112 (3). Pendant 112 (3) may also be worn as a belt-clipped TD, a wrist-worn TD, or another type of wearable TD based on the availability of accessory parts adapted to hold or retain the pendant in a physical form for wearing. Pendant 112 (3) is adapted in this embodiment as a Bluetooth™ enabled tracking device that may be paired to client-operated smart phone 109 which, is also Bluetooth™ enabled for short range wireless communication.

A client operating smart phone 109 aided by SW 113 may also have access to one or more other electronic devices 112 (1-n) like an electronic weight scale 112 (1). Weight scale 112 (1) is adapted as a smart weigh scale having a capability of weighing total weight, body mass index (BMI), bone density, and can separate lean body mass from fat. Weight scale 112 (1) is enabled for Bluetooth™ and can communicate over a Bluetooth™ network with a client operating smart phone 109. Another device that may be provided to a client operating smart phone 109 is an electronic bottle 112 (2). Weight scale 112 (1) may provide data that is not trackable using a pendant.

Bottle 112 (2) is a Bluetooth™ enabled device that a client operating smart phone 109 may use while engaging in activities that may be tracked such as by using the pendant as a tracking device. Bottle 112 (2) may communicate with smart phone 109 using BT L Bottle 112 (2) is adapted by sensor to report volume of liquid that passes the cap portion of the bottle. The bottle may indicate loss of volume periodically; receive notification from smart phone 109, among other capabilities. In this embodiment, a smart blender is provided referenced herein as device 112 (n).

Blender device 112 (n) is also Bluetooth™ enabled and therefore may be paired with from smart phone 109. Blender 112 (n) may receive recipes and provide instruction to a user about how to make the recipe. Blender 112 (n) may be adapted to track a client's prepared nutritional liquids and may include both manual and touch display command interfaces. Blender 112 (n) includes at least one child proof protection mechanism to prevent children from operating the machine (prevents blades from spinning). In one embodiment blender 112 (n) may recognize images and recipes fed to it and may automatically blend at the correct speeds and lengths of time to produce a desired liquid or semi-liquid product.

In one embodiment, a client operating smart phone 109 may have access to one, a combination of, or all of devices 112 (1-n). In a general embodiment, the client must use at least the pendant device 112 (3) for activity tracking and may optionally add another device like bottle 112 (2), scale 112 (1), or blender 112 (n). Each device produces its own unique device-specific data and general data of interest to the client. It is desired that more than one device may be used in combination to produce unique data sets that may be uploaded over Internet 101 to a data processing and client fulfillment and recommendation service such as working domain 102.

Working domain 102 represents the back-end part of the tracking and recommendation service in this embodiment. Working domain 102 may be a set of reserved cloud servers and repositories and data processing tools (SW) that are strategically adapted to provide a stable base history of the health of a client and baby including making recommendations to the client, and fulfillment services for the client for products like vitamins, electrolytes, foods, proteins, micro-nutrients, diapers, lotions, baby products etc.

Working domain 102 includes a local area network (LAN) having connection to Internet 101 through a data network hub 108 such as with an Ethernet network for example. LAN 103 supports a data processing server 106 labeled a user tracking server (UTS). User tracking server 106 hosts a SW application 122 adapted to receive aggregated data, normalize that data for processing, and processing the received data to results that may be maintained in a server-connected data repository 120 adapted to contain client data formatted as user labs data. User labs data may include but shall not be limited to the current levels of nutrients a client is taking, the statistics (base state) of the client relative to nutritional health, and hydration, the statistics relative to a client's weight, BMI, etc. (base state). This data may be goal-oriented data where the tracking service cooperates with a recommendation service, referenced herein as recommendation SW 123 hosted on a recommendation server 107. Such cooperation may result in improvement in the client's nutritional and electrolyte intake and in the recommendations certain activities to the client to help the client attain one or more goals relative to health and fitness in general including the health and fitness of one or more than one baby being carried to pregnancy term by the client.

User tracking server 106 may receive data from a client such as one operating smart phone 109, for example. In one embodiment, information server 105 and website 116 function as a proxy and may broker the connection between cloud-based services and the client device. Client device 109 running SW 113 may access all the tracking data from pendant device 112 (3) over an established Bluetooth LE™ connection requiring device paring and discovery. In one embodiment, pendant 112 (3) may automatically send all its tracking data whenever the wireless connection is active between the pendant and smart phone. Tracked data may include motion data including steps or strides taken, direction of or route of travel (GPS), heart rate, respiratory rate, blood pressure rate, local ambient temperature, and exposure levels to ultraviolet radiation (UV).

The client application may also take data from any other device the client is using as they are all wirelessly enabled for Bluetooth™ technology. Bottle 112 (2) may provide hydration rates over a tracked period, bottle refill requirements, and temperature of the liquid. Scale 112 (1) may provide weight data separated into categories such as a body mass index, lean mass, bone density, muscle tone, visceral fat count, etc. In one embodiment, bio-electric impedance analysis (BIA) is incorporated into the scale top surface and circuitry to enable calculation of overall weight, total body water (TBW), lean fat-free mass, visceral fat, overall BMI, bone mass and density. Blender 112 (n) may provide historical data of blender use events including recipe titles and data about the ingredients used in the blender and the recommended steps for creating a blended product. In one embodiment, the service may calculate how efficiently raw products purchased by the client are used and with what frequency or regularity.

It is noted herein that a pregnant client operating smart phone 109 may practice the invention with only the pendant, the smart bottle, and the client application without departing from the spirit and scope of the invention. It is also clear to one with skill in the art that the client may use all the illustrated devices or any combination thereof including the pendant device. In one embodiment of the present invention, all the device data is uploaded to the smart phone 109 through application 113 and Bluetooth™ connectivity. Data aggregated from the sum of devices including the pendant device may be uploaded to UT S 106 running SW 122. The data may be received on behalf of a client and normalized for processing. In one embodiment, calculations are made across the aggregated data to produce any results that were not clear in the data.

In one embodiment, SW 122 may access third-party data from one or more local or regional information sources such as a weather service, for example, to get temperatures, predictions, humidity levels, pollen levels, particulate levels, which may influence the immediate health of a client being tracked. A third-party server (TPS) 104 is depicted supported by Internet 101 and connected to a data repository 118 adapted to contain third party data. In one embodiment, discovery of third party information is governed by rule or constraint. In an example, a third-party data set squired during processing of tracked data may reveal very hot temperatures for the trail the client is hiking or has hiked.

Hot temperature along the route may cause more water loss than normal leading to a special recommendation for a measured number of additional electrolytes and maybe a protein bar listed in the user's account as a product the user may have access to or one the user has not purchased. In this case the protein bars may be part of a recommendation that may translate into a transaction made by the client through the client application, the transaction based on a recommendation made to the client after processing data about the client. The processed data may be saved on behalf of the client in repository 120 user's labs data. The fresh data in repository 120 may also be processed in comparison to user lab's history of the same data reproduced over time. Therefore, data in user's labs may be refreshed daily or at will of the client.

SW 123 outputs the resulting processed data to recommendation server 107 running recommendation SW 123. Recommendation SW 123 is adapted to read results received and determine, with additional calculation if necessary, whether any recommendations or special insights or other notifications may be ordered for the client because of a noted shift or deviation of certain data from a base norm data value. A base normal for the pregnant client may be established early within the first trimester of pregnancy through repetitive tracking and adjustment of results. Recommendation engine 123 may create general notifications for encouragement, congratulations on goal achievements, tips or advice relative to any data. In one embodiment, SW 123 may participate in chat conversations with a client through application 113 using artificial intelligence (AI), optical recognition for images and text, and interactive voice recognition capabilities (IVR).

Recommendation server 107 aided by recommendation engine 123 may access a base of previously compiled knowledge in data repository 121. Experts in nutritional science contribute to and may provide updates to information held in repository 121. Data within repository 121 may include results and methods of clinical trials and knowledge data resulting of clinical study along with general knowledge data surrounding nutrition where it applies to pregnancy in each trimester of pregnancy. Furthermore, the knowledge in repository 121 may include knowledge data surrounding early childhood. In one embodiment, the service of the present invention extends past the birth of the client's child for another 720 days of early childhood.

Recommendations are therefore tailored to optimum nutritional health of the pregnant mother and fetus during the 280 days of pregnancy and for both the mother and child or children after birth into early childhood. Nutritional requirements change over the course of pregnancy and after the birth of the child. The system of the present invention considers mitigating factors described above and adjusts nutritional recommendations directed to the client. In this way, the system of the invention may make allowances for known conditions such as a current pregnancy. However, the novelty herein extends to potential clients that may have other known conditions to which knowledge about may be previously assembled and accessed to help optimize nutritional health. In the category of pregnant women, there is a market demand of 25 million or so combined from target markets in the US and in China.

The system of the invention tracks proteins fats and carbohydrates (PFCs) including micro-nutrients (vitamins, minerals etc.). The system may track exposure to sun and how much vitamin D is revealed from foods eaten to determine whether a client is deficient or needs more vitamin D. The client application will automatically input the information to figure out your RDA (Recommended Daily Allowance).

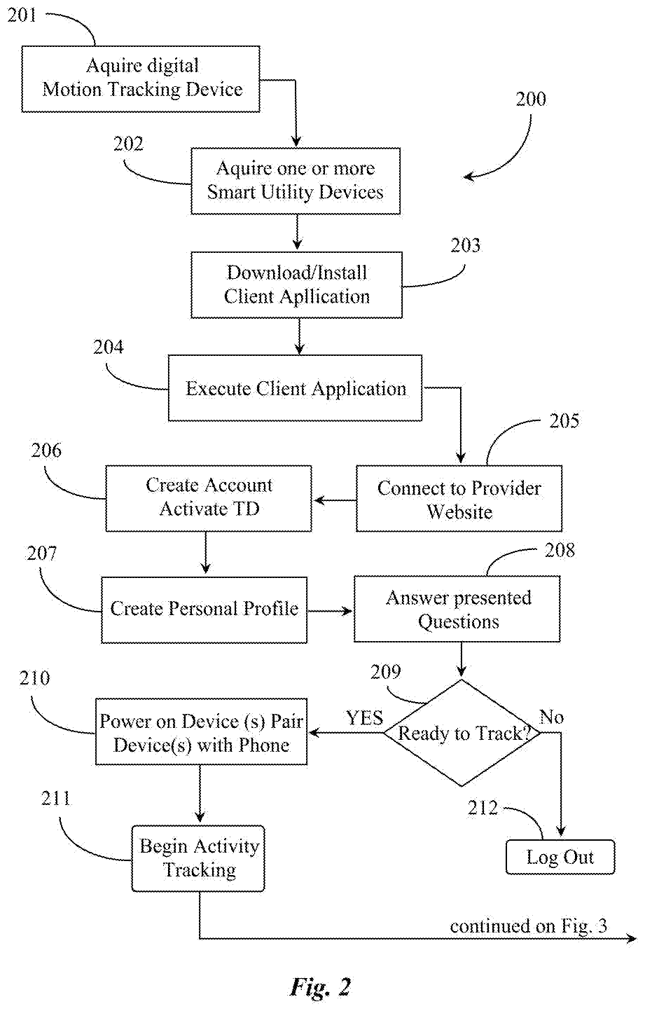

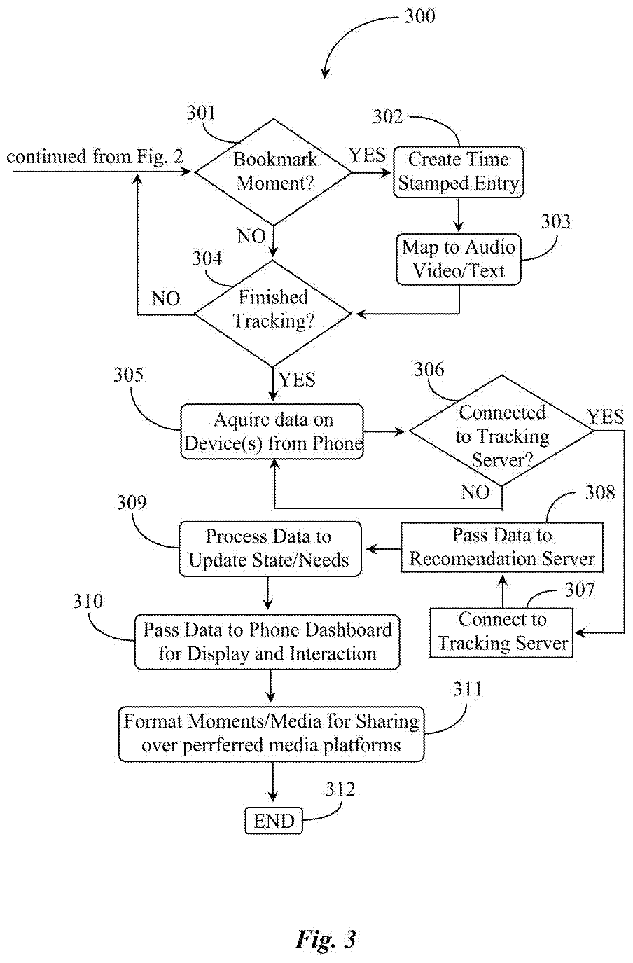

FIG. 2 is a process flow chart 200 depicting steps for beginning tracking and recommendation services for a client. FIG. 3 is a process flow chart 300 depicting the process of FIG. 2 continued. Referring now to FIG. 2, as a first step 201, a client may squire a digital tracking device analogous to the pendant device 112 (3) of FIG. 1. In step 202, the client may acquire one or more additional devices analogous to any of the other devices 112 (1, 2, n). In one embodiment the client may purchase a kit containing at least the pendant device, one other device like the smart bottle, and a link to the web site and client application in one example. In one embodiment, the kit may also include previously packaged vitamin supplements in liquid and or in powder or pill forms that are generally engineered to provide optimum vitamin supplementation for a trimester of pregnancy. In one embodiment, the vitamins covering the first trimester of pregnancy may be provided in the kit.

At step 203, the client may connect online through the provided link and download and install the client application. The client application may be installed on an Apple device or an android device. The application is a browser-based interface connecting the client to the service of the present invention. At step 204, the client may execute the installed application and may connect back to the Web site through the client application at step 205. At step 206, the client may create an account with the service and activate the tracking device (TD).

At step 207, the client may create a personal profile for identification by the system. The profile information may include general profile data and the client may grant permission to the system to access established profile information about the client that is published on one or more other online accounts. Information contained in a client profile may be used to help provide recommendations to the client. At step 208, the client may be prompted to answer a series of questions adapted to enable the system to start a base line for the client before tracking. Client height, weight, general diet, general hydration, current vitamins, average amount of sleep, and other data provided by the client may be used by the service to start a client's lab. A client's lab data may be presented to the client through the client application.

At step 209, the client may determine whether to begin tracking for the first time or not. At step 209, if the client does not want to track activity, the client may log off the service and reconnect later. If the client decides to track activity, the client may power on the pendant, which may be worn as a pendant or on the wrist or clipped to a belt in step 210. The client may also pair the pendant device with the client phone through the client application in step 210. Other devices may also be powered on and paired for Bluetooth™ LE or the established short-range wireless scheme that might otherwise be used without departing from the spirit and scope of the invention. It is noted herein that the pendant tracking device may “wake up” if a low powered motion sensor picks up any movement of the device. Once the device is paired it may be automatically recognized by the client phone in future tracking operations. At step 211 the client may begin activity tracking.

Referring now to FIG. 3, at step 301 the client may determine during active tracking whether to create and bookmark a moment. A moment is any point in time during active tracking that a client wants to preserve in time as a defined moment or event that may also be published with accompanying media. If at step 301 the client does not determine to bookmark a moment during tracking, the process simply resolves back to the active tracking which is not interrupted. If the client decides to book mark a moment at step 301, the client may create a time stamped entry by depressing a button on the activity tracker causing the moment to be created with a date and time stamp as well as a location.

In one aspect of creating a moment, the client may also have an opportunity to associate any relative media to the bookmarked time slot at step 303. Relative media may include media provided by a third party like an ultrasound clip, for example, or media created by the client on the smart phone of the client or by an agent of the client on a remote device where that media may be transferred to the client and then associated with the bookmarked moment. In another aspect of the method, the bookmarked moment may be stored on the pendant until tracking has ceased and may be uploaded without media whereupon the service may remind the client of the moment date time and location, and then prompt the client through the client application to then classify the moment and create and or add any media that may be relevant to the moment.

It is noted herein that tracking activity may occur on the tracking device with or without the client's smart phone. If the smart phone is active while tracking is concurring, and the devices are paired for communications, then tracking data may be sent to the smart phone as it is collected. If a moment is created while the smart phone is on and connected, then the client may create notes, video, photos, audio recordings or the like and those files may be automatically mapped or tagged to the last created moment received on the smart phone.

At step 304, the client may determine whether they are finished tracking their activity. If the client is not finished tracking at step 304, then the process simply resolves back to active tracking and if desired more bookmarking. If the client determines they are finished tracking at step 304, the client may acquire the tracking data from the pendant device at step 305, for example, if the smart phone was not on or paired with the device already during tracking. Once the tracking data is acquired through the client application, the client may determine whether to connect online and sync with the server at step 306, if not already connected.

If the client determines not to connect and sync with the service at step 306 then the process may resolve back to active tracking or renewed tracking. It may be noted that the pendant device has enough memory to store tracking data for more than one tracking event. However, it may be recommended that the client sync with the service on a more regular basis such as daily so that the client's lab data may be refreshed, and the client may receive potentially important notifications.

If the client decides to connect to the tracking server at step 306, the client may connect and sync (pass data and receive data) with the server at step 307. At the server, the data collected from the client may be normalized for processing and processed for results in step 308. At step 309, the processed data may be passed to a recommendation server for further processing and recommendation updating. It is noted herein that both the user tracking and recommendation services may be combined as one processing application running on a single machine or may be distributed over more than one machine.

New data may be written to the user's lab data to update states of the client and fetus. Recommendations may be made by the service to the client and presented as notifications to the client. At step 310, the recommendation and or the tracking server may update the user's dashboard (client application) with the updated client state data and any new notifications. In one aspect, the website brokers communication between the processing servers and the client device and may provide client lab data to the client interface when the client logs into the website. It is important to note herein that a notification may be benign to nutritional health but focused more on encouragement of the client or emotional support of the client. A notification may be relative to nutritional health like a notification to add more vitamin D to a diet or a reminder to approve a next order of micro-nutrients.

Notifications may also be predictive based on data that a client has entered like a plan to hike a specific trail where location, temperature, air quality, and the like may be predicted accurately using third party information. Such a notification may inform the client as to how much fluid to bring and or if any protein might be suggested such as two bars for the scheduled hike. At step 311, the client may have an opportunity to classify moments and to format moments and relative media for publishing on a desired platform. In one aspect the client application has a facility to add contacts like close friends and family and may provide those user's online access to the published information.

In one aspect, the moments may be automatically formatted for a public platform the client has an account with like Instagram™, Facebook™, Mixbook™ and other social platforms. In one aspect the client application provides application program interfaces for formatting a moment with media as a POST, or addition to a story board in a way that enables the client to preview and approve the publication of the moment and associated media without a requirement to scroll for the media. The process may end for the client at step 312.

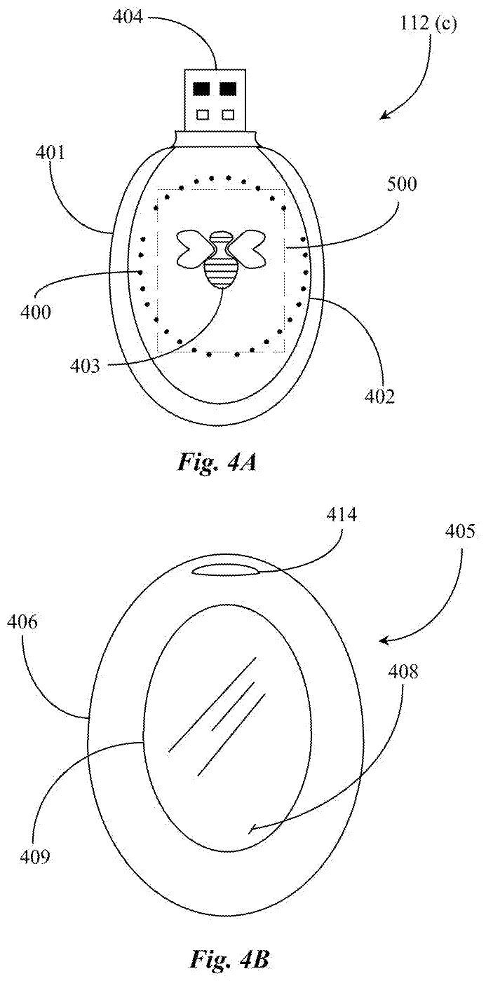

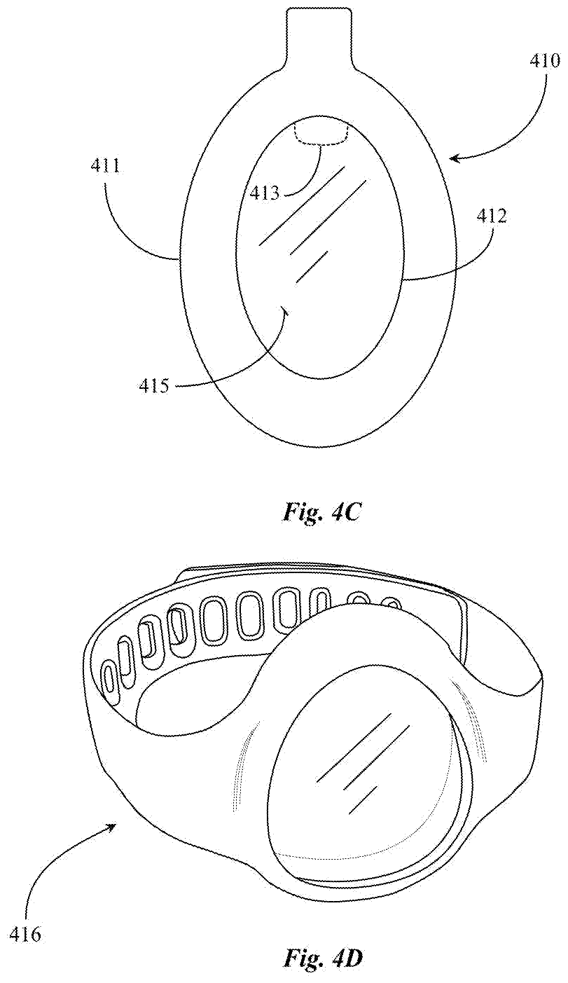

FIG. 4A is a top view of pendant 112 (3) connected to a USB charge cradle according to an embodiment of the invention. Pendant 112 (3) is a pregnant woman's version of a tracking device. Pendant 112 (3) may include one or more rechargeable batteries like a lithium ion battery. Charge cradle 401 uses USB head 404 and includes a charge contact pad set that contact the rear charging contacts on the pendant. In another embodiment, pendant 112 (3) may be magnetically charged. In still another embodiment, pendant 112 (3) may include a small charge port adapted to accept a plug from an AC charging module.

Pendant 112 (3) may be assembled from plastic molded housing parts and contains the required electronics components to enable data tracking functions and offload or communication of tracked data to another device like a client smart phone running the client application. Pendant 112 (3) uses Bluetooth LE™ to communicate data. In other embodiments, near field communication (NFC), radio frequency identification (RFID) infrared, or other wireless short range technologies may be used without departing from the spirit and scope of the present invention. Pendant 112 (3) has an internal space or foot print 500 for housing the electronics components of the pendant.

The front face 402 of the pendant may be manufactured (typically molded) of a durable polymer material. The pendant may be adapted to snap into the charging cradle and may be charged via a USB computer port or a USB port on a charging device. Pendant 112 (3) is purposely designed not to look like a typical tracking device. A user interface (UI) mechanism or indicia 403 is provided in the form of a proprietary logo, which is a bee in this case. UI 403 may be manipulated by the client to at least bookmark a moment.

In one embodiment, UI 403 also enables clearing the device of all tracking data, syncing the device with a smart phone running a client application, and toggling between preset tracking modes that may differ in sensors employed and types of data tracked. In one embodiment, tracking steps on a hike maybe one mode of tracking whereas tracking a client's motion, breathing pattern and heart rate during sleep maybe a different mode.

Pendant 112 (3) may include a pattern of LED micro indicators 400 strategically arrayed about the UI icon 403. In this embodiment, each grouping of ten micro indicators 400 may be dedicated to a single data point, for example one row of ten micro indicators may gauge how many steps were taken towards a goal number of steps, a next row may indicate how many calories have thus far been burned in activity, and the last row may inform a client how many ounces of hydration toward a hydration goal have been consumed. In one embodiment, a client may set up the UI micro indicators to display only data results from a single tracking activity. In another embodiment, the micro indicators may be programed to display a day of results data compiled in more than one tracked event, or a week of tracking data. However, it is suggested that regular periodic syncing with the smart phone and the service be daily so that deviations from baseline normal are available sooner.

FIG. 4C is a top view of a polymer jacket 405 adapted to hang the pendant device about the neck in typical pendant wear position. Jacket 405 may be molded from a flexible but durable polymer material 406 that may be UV resistant. Jacket 405 includes a back wall 408 and a substantially centered opening 409 through which the pendant device may be inserted for practice in pendant mode. Jacket 405 includes a pendant hang loop 409 adapted to accept a chain or tether for hanging the pendant.

FIG. 4C is a top view of a polymer jacket 410 adapted to clip the pendant onto a belt worn by the client. Jacket 410 may be a molded polymer piece and includes a clip 413, which may or may not be plastic or a contiguous part of the jacket. Belt loop jacket 410 has a back wall 415. Jacket 410 may be molded from a durable poly material 415. Material 415 may be analogous to material 408 of FIG. 4A. Pendant 410 has a substantially centered opening 412 through which the device may be inserted for wearing the device on a belt worn by the client.

FIG. 4D is a front perspective view of a flexible wrist band jacket 416. Jacket 414 is adapted to accept the pendant device for wearing on a client's wrist in the fashion of a typical wrist worn tracking device. Jacket 416 may be molded from a durable UV resistant polymer material like the other accessory parts described above. Jacket 416 includes a back wall 417 and a centered opening 418 through which the pendant device may be inserted. The wrist flaps connect by pressing molded snap features into molded opening as is typical of flexible molded wrist bands. All the forms provided above may be included in a kit along with the pendant and starting pack of nutrients and the smart bottle for tracking hydration. Other forms may be provided for wearing the pendant in addition to or aside from the forms described herein.

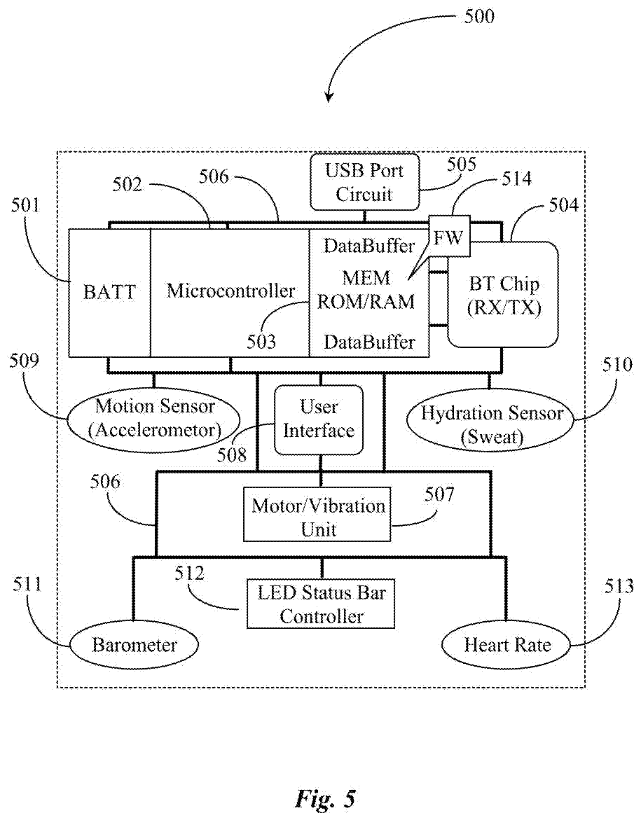

FIG. 5 is a block diagram depicting basic electronics components in footprint 500 of pendant 112 (3) of FIG. 4A. Space 500 may house a battery(s) 501 like a lithium ion battery. Battery 501 powers a micro controller 502. Micro controller 502 has access to a memory block 503. Memory available to the micro controller may include random access memory (RAM), read only memory (ROM), flash memory etc. Memory block 503 may include data buffer sectors for buffering or queuing data during sync operations. A Bluetooth LE™ wireless chip 504 is provided in this example to enable wireless transmission and receipt of data. A communication and power bus structure 506 is provided to logically represent communication pathways and power distribution to sensors and electric motor components.

A USB port circuit 505 may be provided or appropriate charge contacts adapted for cradle charging as previously described relative to charge cradle 401 of FIG. 4A. In one embodiment, data on the pendant device may be uploaded to a computing device having a USB port and cable adapted to plug into a USB charge port like port 505. In a preferred embodiment, the data is transferred wirelessly. A user interface (UI) 508 is provided and connected to bus 506 and to a motor/vibration unit 507. Unit 507 may be activated through the user interface or by the micro controller to vibrate or otherwise agitate to a level that the client may feel it and check state. In one embodiment, motor/vibration unit 507 may be triggered as a result of successful bookmarking of a moment to confirm to the client that the action was successful.

A motion sensor 509 is provided to the pendant circuitry and is connected to bus 506. Motion sensor 509 may be adapted to track motion, speed, direction, and orientation like an accelerometer with a compass or gyro function. In one embodiment, a hydration sensor 510 may optionally be provided that may be adapted to detect and measure electrolytes in the client's skin and measure hydration state. In one embodiment a barometer sensor 511 is provided and adapted to measure air pressure and report elevation. Sensor 511 may utilize a magnetometer sensor for orientation with Earth's magnetic field to provide orientation and an altimeter sensor for determining altitude. A heart rate sensor may be provided to measure the client's heart rate during activity. In one embodiment, an ultraviolet exposure sensor (not illustrated) may be provided on the pendant to gage potential UV exposure of to client wherein recommendations may be made to the client concerning the amount of UV exposure and products available to mitigate that without losing Vitamin D absorption, for example.

An LED status bar controller interface 512 may be provided for maintaining a LED powered interface like status bar array 400 depicted in FIG. 4A. A client may look to the display of LED micro lights to see how many steps were taken (stride, distance), how many calories were burned, and how many ounces of hydrating fluids were consumed. This example includes the ability to display recent progress on the face of the device through each 10 dot LED row. If for example a goal for the client is walk 1000 steps, then each individual LED dot confirms one hundred steps take so if the client took 300 steps of the goal three of the micro indicators would illuminate. In one embodiment the client may view the same interface in the client application and see what the progress indicators look like at any given time.

Firmware (FW) 514 is provided as an executable instruction set for general operation of the tracking device. In one embodiment, motion sensor 509 may reside in a low power consumption state when it is not active like a sleep mode. In this aspect variation, the sensor may boot the micro controller when movement by the client putting on the pendant, for example is sensed. Booting the micro controller may be followed by activating the sensors for tracking data and making the pendant device discoverable on the Bluetooth LE™ network. In one embodiment the pendant device automatically sends tracked data directly to the client application if both devices are connected wirelessly.

In one embodiment, there may be a physical power toggle switch or on/off switch on the pendant device. In one embodiment, a client may boot the pendant device remotely using a signal command that toggle the low power sleep state of the micro controller to waking mode. In another embodiment, there may be a small audio interface for recording simple voice input from the client. For example, if a client creates a moment, that client might also say a few words in a microphone to classify the just created moment. In this way some interaction may occur between the client and the pendant that does not require the client to have the smart phone on and the application running. In still another embodiment, there may be a carbon particulate sensor and or smoke particulate sensor (not illustrated) on the pendant that may calculate air quality during a tracked activity.

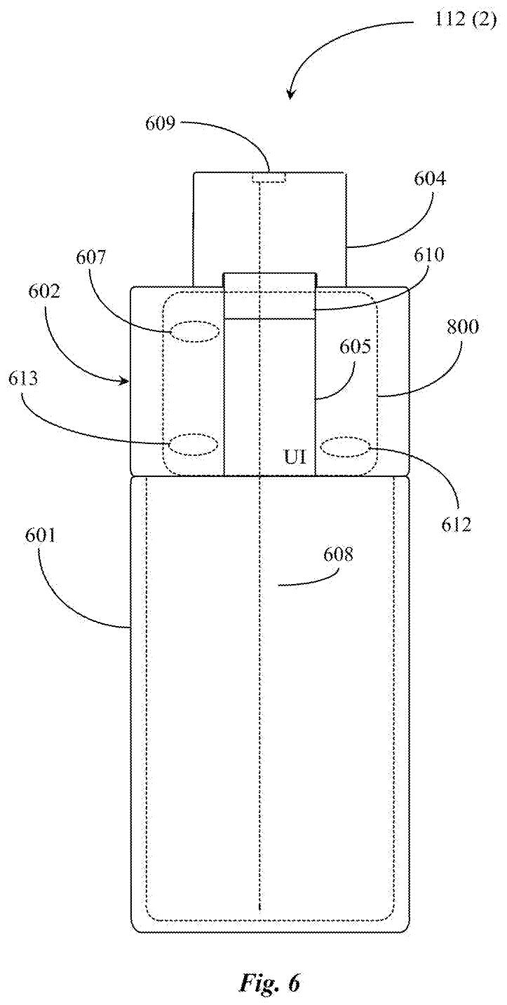

FIG. 6 is an elevation view of smart water bottle 112 (2) of FIG. 1. Bottle 112 (2) is a Bluetooth™ enabled device that a client operating smart phone 109 may use while engaging in activities that may be tracked such as by using the pendant as a tracking device. Bottle 112 (2) may communicate with the client smart phone using Bluetooth or another supported wireless protocol. Bottle 112 (2) is adapted by sensor to report volume of liquid that passes the cap portion of the bottle. The bottle may indicate loss of volume periodically; receive notification from the client smart phone smart phone, among other capabilities.

A pregnant client is hydrating for two persons, themselves and their babies. As a client progresses through each trimester of pregnancy hydration requirements and goals may shift. Connected bottle 112 (2) includes a sterile stainless-steel container of about a 17-ounce volume. The container portion is male threaded at the top to accept a smart electronic bottle cap 602. Cap 602 includes sealing gaskets. Cap 602 contains the electronics 800 including sensors for tracking hydration rates, and some ambient conditions that may affect hydration, especially for a pregnant woman.

Cap 602 includes a spout 609 connected to a straw 608 that passes through the cap some distance into the container portion 601. Electronics 800 within cap 602 are sealed from the ambient environment and may be protected through water proofing techniques for electronics. Cap 602 includes a top spout cover 604 that may be spring loaded and hinged at one side of cap 602. A retainer plate 610 is included and may lock the top cover 604 over drinking spout 609.

In one embodiment a part of cap 602 may include a spring-loaded twist lock mechanism in line with cover 604 whereby a client may twist the twist lock in one direction to release cover 604 and in an opposite direction to lock it back down. In one embodiment spout 609 is aligned to but not physically connected to a passage through the cap into the container portion. The spout piece may install on a track seal mechanism whereby the spout may be urged over a seal and may be cut off from liquid passage through the cap and urged back to an open position to sip from the bottle.

Bottle 112 (2) includes a water volume flow sensor 607 in this embodiment that may be adapted to measure and log the exact amount of liquid passing through the cap over time of a tracked activity. Bottle 112 (2) may include a temperature sensor 612 for measuring and reporting the temperature of the fluids in container 601. Bottle 112 (2) may be adapted as a thermos bottle having material properties that promote slower temperature change from the original temperature of stored liquids. In one embodiment, a UV (ambient light) sensor 613 is provided and adapted to track a client's potential sun exposure.

A user touch screen interface 605 is provided and located on one side of cap 602 in the form of an embedded display. Display 605 may present indicia like picture icons representing data point like electrolytes consumed, hydration rate in ounces, temperature of liquid in the container, UV exposure level, ambient temperature, and a flashing or vibrating icon reminding the client to drink from the bottle. The server application processes data received from the water bottle through the client application and may rely on general third-party information such as geographic location as determined through the client's GPS capability to gather knowledge that may not be immediately available from a device sensor or that can be utilized to corroborate the accuracy of a device sensor.

Weather information including humidity levels, particulate levels pollen levels, during tracking activity may cause recommendations to be made by the system during the tracking activity or immediately after the tracking activity to maintain base line goals. In one embodiment, a client may schedule an activity where the bottle will be used like a hike, for example, and may request that the system estimate the appropriate rate of hydrating (taking sips) from the bottle and may recommend packing more than the volume of water or liquid in the bottle based on planned distance, difficulty level, weather and temperature forecast for the estimated time of the hike.

Recommended daily hydration rates and pregnancy vitamins and mineral allowances may shift during progression of the woman through each trimester period. Calculations take into account the needs of the fetus nutritionally in addition to that of the mother. The pregnant client's lab data may be accessed by the client through the client application and presented to the client on a summary page dashboard screen. The devices like the pendant and the bottle may sync with the client application and receive data for convenient display on the devices so that the client may see progress against a goal on the device instead of being required to access the application such as while hiking for example.

One with skill in the art will appreciate that use of more than one dedicated device such as the described tracking pendant and the connected bottle for example, provides unique data points that may be combined in the tracking data and processed along with third party data and previously provided client data not to mention repetitive client use history data reflecting the client's average baseline levels for general fitness state and where those levels are relative to goal levels set through recommendation, coaching, and client desire.

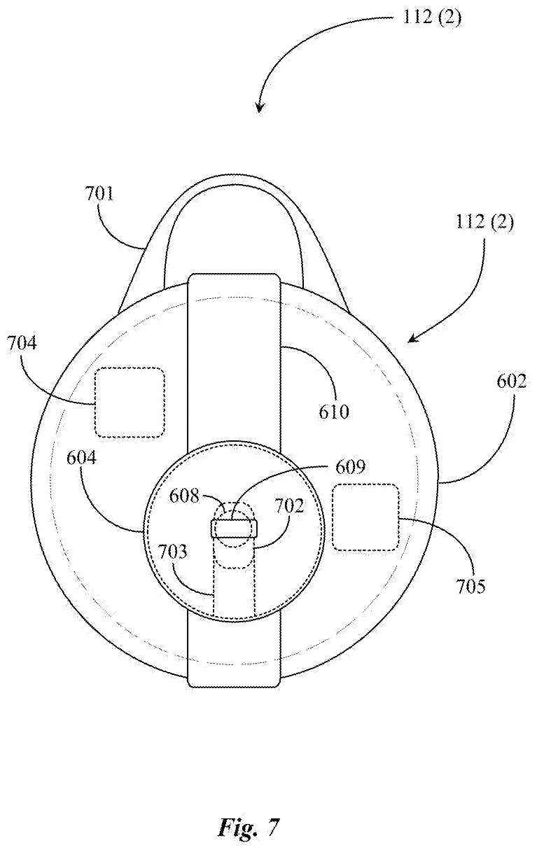

FIG. 7 is a top view of bottle 112 (3) of FIG. 6. Bottle cap 602 includes a hard-molded loop handle 701. Spout 609 may be a slidable piece mounted on a sliding base 702 installed in a track 703. Spout 609 may be positioned along the track to align with passage 608 or moved along the track to seal the spout off from the passage. In one embodiment, an embedded display or more than one dedicated embedded display may be provided in the top surface of the main cap body for convenient display of data or notification to a client operating the device. In this case a display 704 is provided and a display 705 is provided. Displays 703 and 704 may be touch screen displays that may present Icon indicia for the client to interact with through touch to display certain data points to the client.

In one embodiment, a pregnant woman may be in a Pilates class tracking during the late first trimester wherein the pendant may communicate tracking data relative to the exercise movements, breathing rate, heart rate, location, and the bottle may communicate hydration tracking data (amount and rate over time of hydration of the client). The service processes the data in combination with third party data and previously established knowledge data. After a session of tracking the client may update with the service of the present invention and pass the data off to the server for processing results updating and recommending.

A recommendation may be sent to the client application dashboard and over the wireless connection to the bottle for example to increase hydration during tracking of the subsequent session of similar length and intensity. A flashing icon to refill the bottle may be displayed. A reminder to hydrate through notification may be programed into the instruction set firmware on the device. In another embodiment the bottle may receive intelligence from the client smart phone if the application is working and server-connected during the tracking activity. Processing and updating may be performed continuously or periodically over the time of subscription by the client.

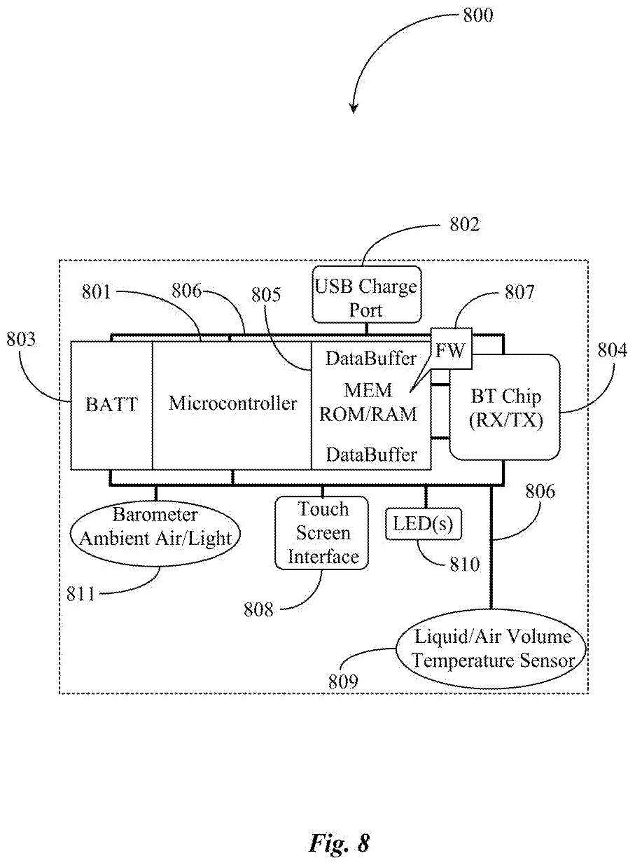

FIG. 8 is a block diagram depicting basic electronics components 800 contained in cap 602 of FIG. 6. Cap 602 may include a micro controller 801 powered by a rechargeable battery 803. Micro controller 801 has access to a memory block 805 adapted to hold ROM/RAM and buffer memory for caching and queuing data. Bus 806 is provided and logically represents data command paths and power distribution paths. Firmware (FW) 807 may be provided as an executable set of instructions for enabling the functions and order thereof enabled by the circuitry and data compiling capabilities of the water bottle. A Bluetooth™ wireless chip 804 is provided to enable device discovery and communication with the client smart phone through the client application.

Components 800 may include a USB charge port enabling charge of the batteries. Like pendant 112 (3), bottle 112 (2) may have a sleep mode and a motion sensor that is adapted to wake up the bottle when it is picked up for use. In one embodiment, a client may discover and wake the device from sleep mode using a wake command to the power mode controller. In another embodiment the water bottle may have a power switch so that it may be physically toggled between power states. In one embodiment, a client has control over current power settings of the devices and may invoke or configure desired levels of power use conservation.

Cap components 800 include at least one touch screen user interface depicted herein as interface circuitry 808. Embedded displays are depicted in FIG. 6 and FIG. 7 with interfaces 605, 704, and 705. There may be sensors provided for measuring and logging air quality, ambient light, and barometric pressure grouped herein as sensors 811. Light emitting diodes (LED) 810 may be provided for back lighting a display or for general illumination where required or desired.

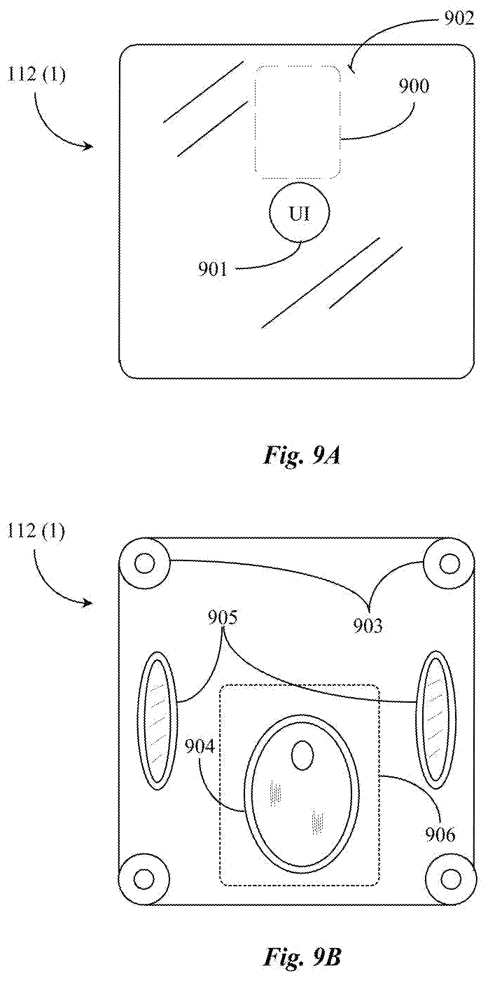

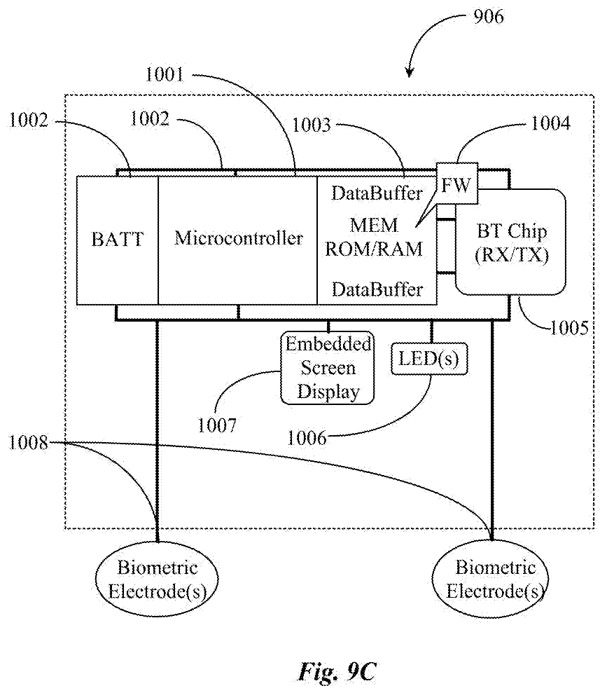

FIG. 9A is a top view of scale 112 (1) of FIG. 1. Scale 112 (1) may be described as a smart scale adapted to measure total body weight, total body fat, water content, bone mass, bone density, and visceral (abdominal) body fat percentage to reveal lean muscle weight. A pregnant woman going through three trimesters of pregnancy may use scale 112 (1) to stay on top of weight issues and water retainment issues that are common for pregnant women.

Scale 112 (1) utilizes an embedded bio electrical impedance system for determining the subset data such as water content, bone density and mass, and fat weight versus lean muscle weight measurements. The scale device is enabled for Bluetooth™ communication with the client's smart phone and provides data to the client application along with the pendant and water bottle. As a client adds devices the discovery of calculable variables becomes much more granular. Scale 112 (1) may be manufactured from a durable polymer and may be covered on the scale top surface with tempered glass material 902. The scale is about a foot or so square and just over an inch or so high.

Scale 112 (1) includes an embedded display 900 that displays data when a client steps on the scale. A toggle switch may be provided to toggle between pounds and kilograms for example. In one embodiment, scale 112 (1) uses standard AA batteries and does not need periodic recharging. However, in one embodiment the scale may use rechargeable batteries like the other devices. The tempered glass material may be fastened to the top surface of the scale via a center mounting hardware represented herein as annular feature 901.

FIG. 9B is a bottom view of scale 112 (1) of FIG. 1. Scale 112 (1) includes ergonomic handle features 905, one on each side enabling a client to carry the scale using either feature as a handle support. Scale 112 (1) includes four riser feet 903 that may be height adjustable to provide leveling functions and cushion from the floor. Scale 112 (1) includes a battery compartment that is removable for inserting batteries and accessing scale electronics including a toggle switch for toggling weight standard presentation. Internal electronic components may occupy a footprint or space compartment 906. Scale 112 (1) is not a tracking device per say and measurements recorded at the scale may be transferred to the client's smart phone application in real time with the wireless connection active or periodically if the client syncs with the scale using the application.

Measurements taken by the scale are used as data input in data processing for recommendations and for making adjustments to fitness data and current supplement needs of the pregnant client and fetus. In one embodiment, the functionality of bio electrical impedance in the scale may be used with additional calculations at the server after data is uploaded to make reasonably accurate estimations of baby weight, and amniotic fluid levels. These estimated values may be subtracted from the pregnant client's weight totals to provide compartmentalized data for both the mother and the child. In one embodiment, a toggle switch may be provided that hides the weight data from display but enables the results to display in the privacy of the data application on the client device. In one embodiment, an automatic setting may be configured to ignore pregnancy state relative to bio-electric impedance functions and calculations when someone else not recognized as the pregnant client is using the scale wherein the scale automatically recognizes the pregnant client and takes bio-electric readings in a pregnancy mode.

FIG. 9C is a block diagram depicting basic circuitry components in space 906 of FIG. 9B. Like other devices described above, scale 112 (1) includes a micro controller 1001 powered by four AA batteries 1002. A power and data bus 1002 are provided for connectivity and data reporting. Micro-controller 1001 has access to a memory block 1003 with ROM/RAM memory and data buffer (cache and queue) memory. Scale 112 (1) includes a Bluetooth™ LE chip for low power syncing with the client application. Scale 112 (1) includes a firmware (FW) 1004 comprising sets of instructions for operating the scale functions. UI screen display circuitry 1007 may enable one or more than one embedded display.

LED circuitry 1006 may be provided to illuminate or back light the embedded display screen. In one embodiment the visual display of data by the scale may be configured according to the preferences of the pregnant client to display result not normally calculated for another user not pregnant. In one embodiment a scale may be a shared device between two or more pregnant mothers wherein each user is recognized instantly when they step on the scale. Identification of a client may be made by initial weight reading and comparison to documented lab data. In another embodiment, the scale may be adapted to measure the footprint of a user and associate that with the user's lab data so that the next time the user steps on the scale he or she is instantly recognized.

Scale 112 (1) uses bio electrical impedance to compartmentalize readings for the pregnant client. A pregnant client may step onto the scale with bare feet to enable bio electric current to flow into the client's body and the scale calculates the weight and water content by first reading the impedance of the current flowing through flesh, fat, bone, and water. In one embodiment the entire space of the top surface of the scale is adapted with bio electrical impedance electrodes distributed across the surface so that a client may step anywhere on the surface and have the scale perform in the impedance mode. It is noted that the impedance mode may be overridden if the scale does not recognize a person on the scale or if a client has disabled the feature. Benefit to a pregnant client over a standard user having preset goals in weight gain or loss are apparent considering the ability of the system to process the data to produce compartmental results attributed the mother and attributed to the baby.

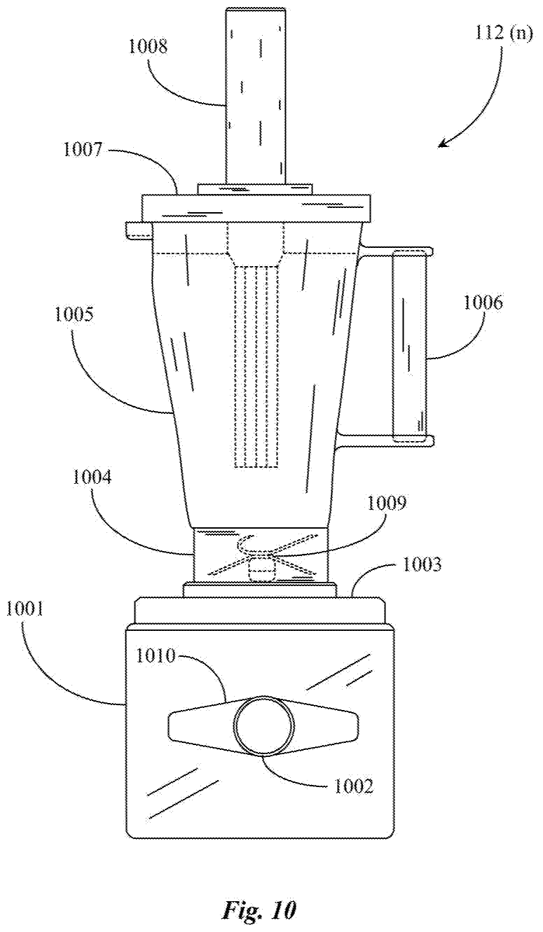

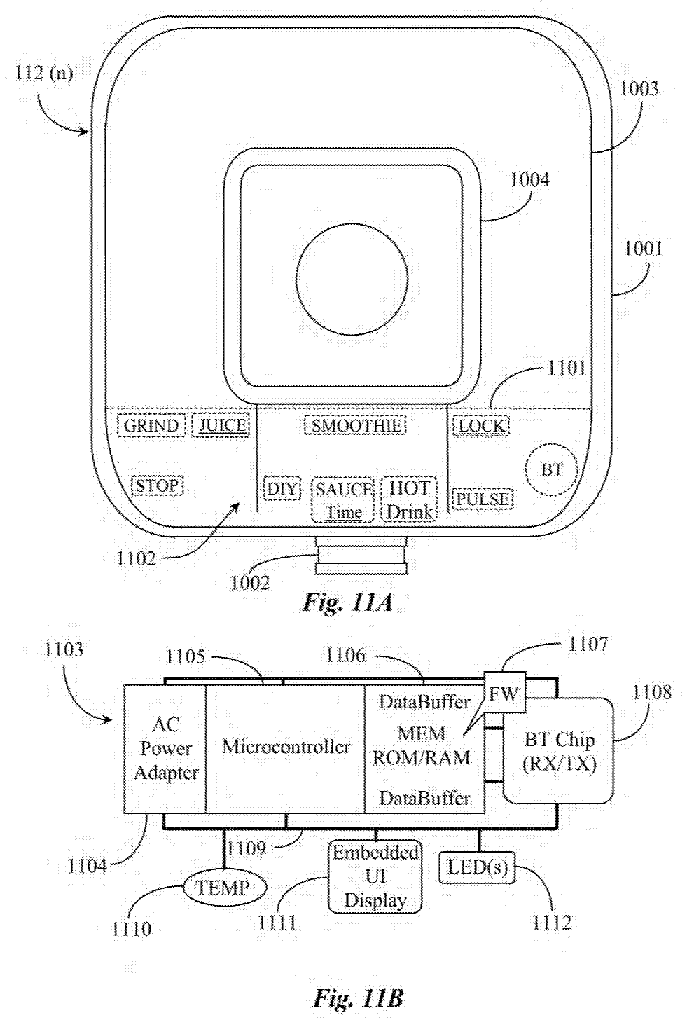

FIG. 10 is a front elevation view of the smart blender device 112 (n) of FIG. 1. Blender device 112 (n) includes a base unit 1001. Base unit 1001 is adapted to contain a multiple speed motor (not illustrated) that drives the blades. Base unit 1001 also includes the electronics components required to enable Bluetooth™ communication with the client application. Blender 112 (n) is adapted for optical recognition of images and text. Blender device 112 (1) may be adapted for voice recognition of the client's voice for voice commanded preparation.

Base unit 1001 includes a manual knob 1002 for blender power on/off, and motor speed selection. Unit 1001 includes an embedded screen display 1010 that may display options for controlling the blender, for example voice control or manual control, or OCR mode for reading and then automatically blending recopies. One important use aspect of blender 112 (1) for a pregnant client is that it may be used in accordance with the other devices to create and store unique recipes for nutritional blends of fruits, vegetables and proteins that are recommended by the system based on system knowledge and results processing relative to the tracking data including the metabolic data, the hydration data, and the body composition data (weights, bone mass, etc.). Recommended blends may also consider goals of the pregnant client through pregnancy and after pregnancy.

A goal of the pregnant mother may be to maintain healthy weight through pregnancy, maintain sufficient energy and nutrition through pregnancy, and increase muscle strength during pregnancy to prepare for birth. Certain blends may be supplemented with recommended nutrients especially prepared or mixed for pregnant women. Blender device 112 (n) includes an interfacing riser 1003 for interfacing with a blade collar 1004 supporting blender blades 1009. Blades 1009 are specially designed to optimize the efficiency of blending from chunking and rough blends to purees or fine blends. Each blade 1009 is serrated and at least one blade has a bend designed to draw in materials to the blades. All parts of blender 112 (1) are free of bisphenol A (BPA).

Blender container 1005 includes an ergonomic handle 1006, a pour spout oriented opposite the handle and a container lid 1007 adapted to accept a special blender spatula tool 1008 for occasional swabbing of the blended materials from the walls of the container toward the center of the container. Mixing tool 1008 may be adapted to measure the temperature of the blended materials. Blender 112 (1) includes child safety features that prevent the blender from turning on without all the components in place, for example, the cap must be down properly on the container, the mixing tool must be down properly on the cap, and the blades must be properly engaged with the motor. Removing the cap or mixing tool from the cap breaks circuit from power to the blender motor and stops the motor. Blender 112 (1) is a commercial grade plug in device requiring AC/DC power.

FIG. 11A is a top view of blender device 112 (n) with the blender container and blades removed. Depicted in this embodiment, are the blender base unit 1001, the interface riser 1003, the blade collar interface 1004 and the manual blender speed control knob 1002. A user interactive touch screen display interface 1100 is provided in the top surface of interface 1003. UI 1100 displays touch interactive control indicia 1102 including a grind selection, a juice selection, and a stop selection. Indicia 1102 further include a smoothie selection, a do it yourself (DIY) selection, a sauce/time selection and a hot drink selection. Indicia 1102 further include a lock function selection, a pulse selection, and a Bluetooth™ activation selection.

In one embodiment, blender 112 (n) may be adapted to recognize digital recipes sent to it over the wireless network from the client application. The blender may assemble the blending functions required for blending the recipe from an on-board repository as may be directed by firmware on the device. Blender 112 (n) may also be adapted to voice command.

FIG. 11B is a block diagram depicting basic electronics components 1103 of blender 112 (n) of FIG. 1. The circuitry 1103 may include an AC/DC power adapter module 1104, a micro controller 1105, memory block 1106 including ROM/RAM and buffer memory, and a Bluetooth™ chip 1108. Firmware 1107 may be provided as executable from memory and includes instruction sets for enabling blender functions and capabilities. A logical bus structure provides connectivity for power and communication between the components.

Voice and optical recognition modules are not depicted in this example but may be assumed present in one embodiment. Components 1103 may include a temperature sensor 1110, which is physically provided in the special mixing tool 1008 of FIG. 10 and measures the temperature of the blended content. Components 1103 may include circuitry 1111 for enabling one or more interactive touch screen displays, and LEDs 1112 for illuminating displays and confirming power on state and the like.

A goal of the present invention is to provide pregnant clients with a service that seeks to optimize fitness during pregnancy (mother and child) through nutritional supplement and vitamin mix engineering and recommendation and order fulfillment of the engineered products. A further goal of the invention is to provide timely coaching of the mother and promote peer and family support for the mother through the celebration of “moments” created by the mother during activity tracking.

The mother may have goals in mind that the system may aid with through recommending nutritional products and activities that are safe for the mother and child and by providing stable and accurate baseline labs for the pregnant client through the first three trimesters of pregnancy and the first 720 days post birth into early childhood. The mothers' needs in both periods pregnant and post pregnancy may be shaped by the many changes in routine, hormones, immunity state, sleep periods, that the mother naturally goes through over 1000 days or so that would not occur in a normal athlete pursuing a normal fitness goal like gaining lean mass. The mental state of the mother is encouraged through positive results, lifestyle and fitness coaching, and peer and family participation from at least a social media perspective.

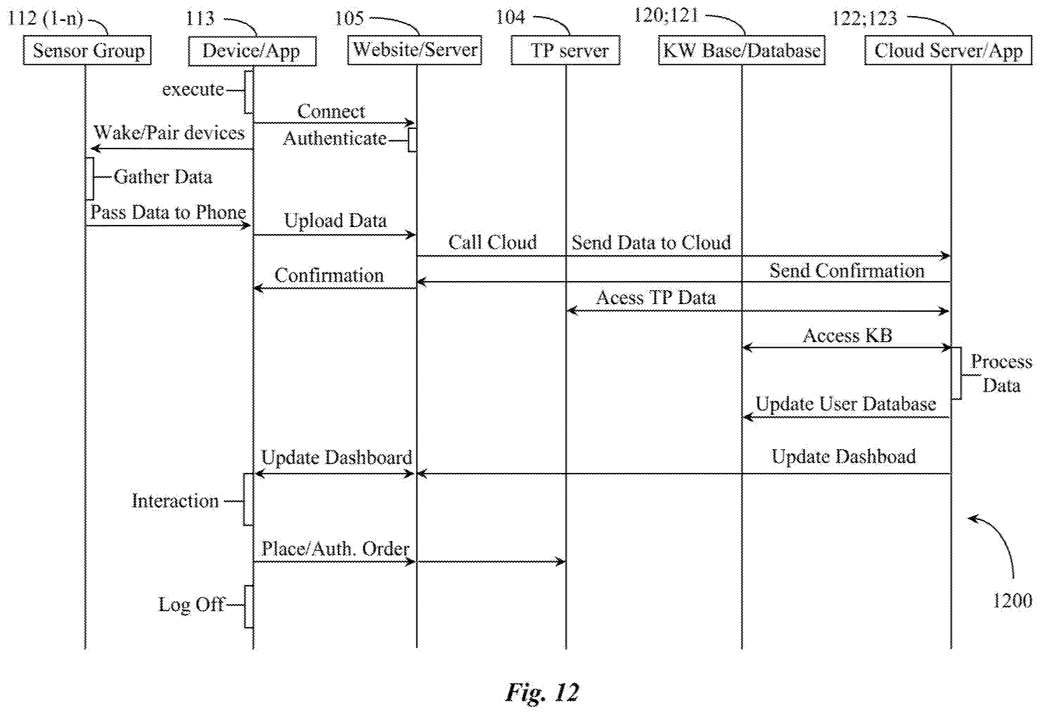

FIG. 12 is a sequence diagram 1200 depicting basic network interaction between the components of the system of the invention. Sequence diagram 1200 includes sensor group 112 (1-n), client application 113, website server 105, a third-party server 104, KW base and labs database 120; 121, and processing and recommendation applications 122; 123.

In one embodiment, a pregnant client may begin practice of the invention by executing the client application 113 from the client smart phone. The client may through the executed application connect with the web site and authenticate to interact and sync dashboard data and receive any notifications or new recommendations. While a client need not be connected to a website or have the client application running on the smart phone while tracking data, this sequence assumes tracking with the phone on and application running in the background.

The client may wake and pair the pendant for tracking and perhaps the water bottle for managing hydration during a tracked activity. Physically moving the pendant may wake the pendant from a low power mode to a fully functioning tracking mode with active wireless connection. The devices may gather and log tracking and hydration data and may pass that tracking data over the wireless network to the client application. The client application may upload the data received from the devices to server 105 functioning as a broker that calls the back-end service and passes the received data to the server for processing on behalf of the client. A confirmation of the request may be returned to the web service (proxy) and back to the connected client application.

During analyzing and processing data on behalf of the client, the cloud-based service may access third party (TP) data from server 104, which may represent any third-party data source like a GPS server, weather forecast server, mapping server, or other public data source servers available on the network. Third party location data may be most important in determining location of an activity and enabling other data such as weather data to be accessed for a location at a specific time. Factors such as heat and humidity levels may affect hydration requirements during an activity.

In one embodiment, a pregnant client may plan an activity and publish the planned event for processing to determine recommended water amount to bring and weather a protein bar should be available, etc. Predictive processing may enable a client to see the number of steps, distance, and estimated calorie burn before engaging in the activity. Since the activity is tracked, the real data may be added to the client's labs data and discrepancy between the predicted results and the real results may be noted to fine tune the predictive calculations.

The tracking and recommendation servers may access KW base and the user labs history data held in repository. The knowledge base comprises information compiled by professionals in the field of nutrition and pregnancy, the information having been rigorously vetted before use by the system of the invention. These professionals having accepted degrees in their fields of study continue to add to the base of knowledge including addition of results of clinical studies, basic medical complications and how they may affect nutrition levels, basic knowledge of the available nutrients, supplements, and electrolytes, and information relevant to physical and mental states and conditions that might affect pregnant women. These professional contributors may also share science articles, tips for pregnant moms, video/audio content, encouragement, etc. in the client smart phone application 113.

All the aggregated tracking data may be processed to produce calculated results. Simple straight forward calculations like the number of calories burned during a tracked activity can be made without consulting additional knowledge, however making recommendations to client's to alter vitamin mixes, increase or decrease protein intake, change amounts of supplements shipped based on existing purchase orders, wear more sunscreen, add more electrolytes, increase regular sleep time, etc. rely on both the tracked data history, third party information, client's current baseline data, and knowledge found in the knowledge base.

After the core application processes data, the results of processing may be added or updated to client lab data held in the cloud on behalf of the client as client baseline history. These same results of processing are forwarded to the web server 105 to update the client dashboard (result summary view) on the client's smart phone application 113. Once the data is accessible through the website, the client may sync with the site through the application to see and to have the opportunity to interact with the data to see detail and to understand, in some respects how data was shaped such as by existing knowledge and third-party data, for example. In interacting with the displayed data and any recommendations, the client may place or authorize orders for products through the application, pay for those orders through the application, and have the products drop shipped to the client's mailing address.

For a pregnant woman with a growing child being carried to term, there may be rather notable differences in system recommended nutrients, vitamins, and food supplements for each trimester of pregnancy. As well, recommendations concerning activities that a pregnant mother may safely pursue may shift in recommended length of activity, intensity of activity, including some activity that may be encouraged by the system such as in the first trimester, but may be discouraged by the system in the final trimester. After a baby is born, the service continues to track the health and activity of the mother now nursing or not and the child may also be considered relative to nutritional health at least relative to general system recommendations for the first 720 days of early childhood. A mother may, in one embodiment, add the child to the activity tracking system as far as adding the child's data along with the mother's data so that recommendations may still be relevant to the child's continued nutritional health.

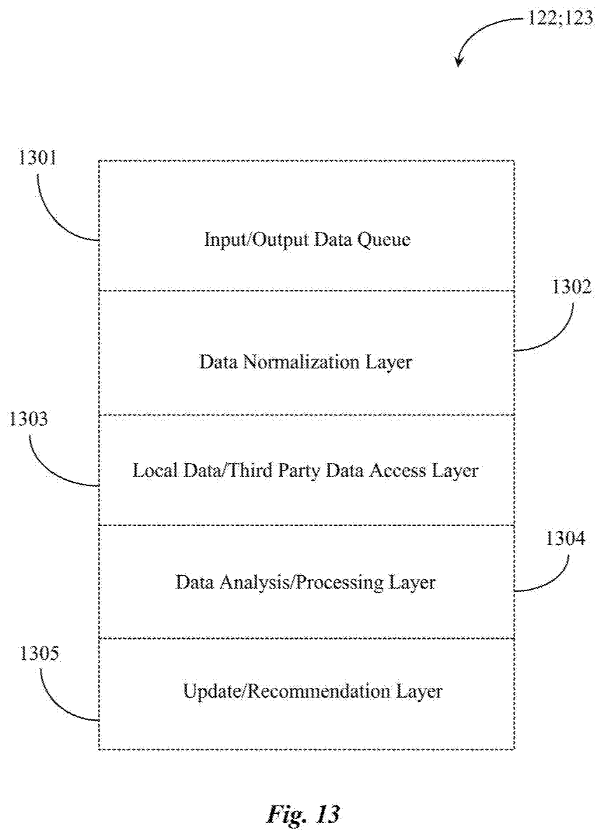

FIG. 13 is a block diagram depicting basic software layers of core service application combining results processing SW 122 and recommendation SW 123. It may be assumed that the main functions of the core application are combined and executed from a single back-end server and that seamless integration with a secure transaction server is implied for product recommendations and client responses to those recommendations. However, the processing and recommendation functions may be distributed over several network-connected machines without departing from the spirit and scope of the present invention.

An input/output data queuing layer 1301 provides for queuing incoming client tracking data received from the client application for subsequent processing, and for queuing outgoing result data, notification data, and recommendation data forwarded to the website for distribution to individual client applications. In one embodiment, the connection between the client and the core application is a direct connection wherein the web site does not retain the client labs data or function as a proxy to the interaction between the cloud and the client. In one embodiment, all the client data stored at the back-end service and stored at the website is encrypted data and not human readable. A unique client decryption token held in the client smart phone application enables the client to read all the data.

A data normalization layer 1302 may be provided to normalize incoming tracking data from multiple client devices for subsequent data processing. Tracking data may, in one embodiment, include data from any cooperating third party devices not proprietary to the service provider, but enabled through SW application programing interface (API) to practice the invention in so far as providing tracked data that may be sent to the client application and normalized at the server after client sync with the server.

A local data and third-party data access layer 1303 may be provided to enable data gathering routines or navigating bots to retrieve relevant data such as location data, weather data, air quality, or other published information that may affect a tracked activity such as rate of recommended hydration for the client. Local data includes client labs history data and knowledge base information. Data retrieved from local data sources may be “data normalized” and used in the processing of the tracked data and for triggering certain recommendations and or notifications to be sent to the client. Recommendations may include purchase recommendations for new products or product amount adjustments for products shipped in existing orders.

A data analysis and processing layer 1304 is provided to analyze and process tracking data. Third party data and locally held client data and knowledge base data may be used as weighting factors in vector processing or comparison analysis to potential trigger of an intervention in the form of a recommendation or notification to the client.

An update and recommendation layer 1305 is provided to enable continuous update of client labs history and to client labs data held at the website and formatted for presentation in the client application dashboard. Layer 1305 is also responsible for generating recommendations or notifications, which are also forwarded to the website and client application. The main layers may be thought of as a results processing engine and a recommendation engine. A rules base containing constraint rules and conditional rules may be provided and accessed during data processing wherein such rules may be considered and may trigger certain stock recommendations based on recent data results calculated for a client.

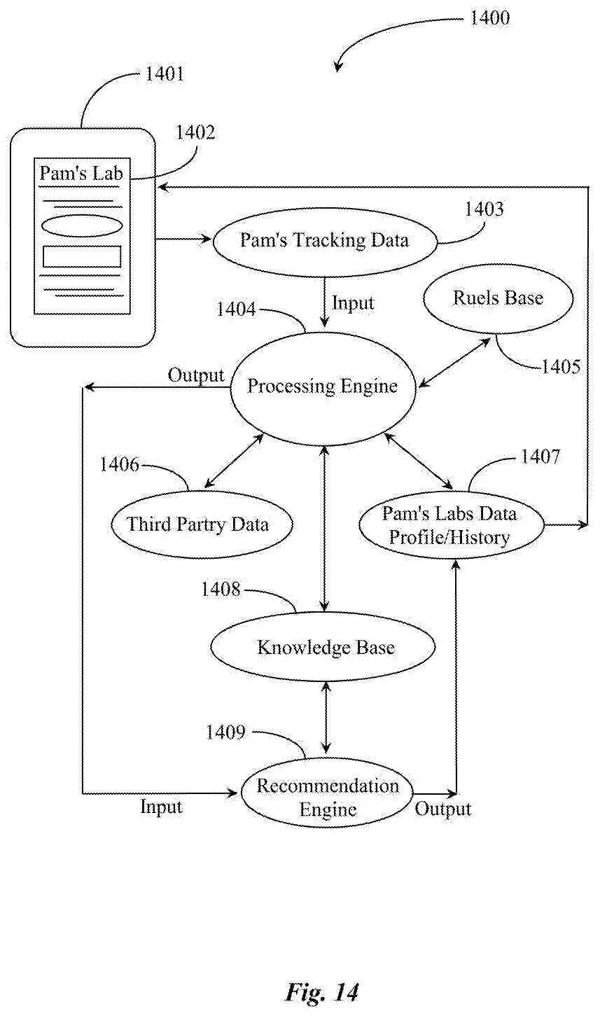

FIG. 14 is a block diagram 1400 depicting functional connectivity between basic components of the service model of the present invention. A client smart phone 1401 running client application 1402 belongs to a pregnant subscriber Pam wherein the application is displaying Pam's labs. Pam is presumed connected to the tracking server through the client application and website. Pam's tracking data is presumed gathered from Pam's tracking pendant and smart bottle, and or other devices and is input to processing engine 1404.

Processing engine 1404, in addition to Pam's tracking data, has access to knowledge base 1408, a rules base 1405, third party data 1406, and Pam's labs data and profile history. The output of processing engine 1404 may be input to a recommendation engine 1409. Recommendation engine 1409 may have access to knowledge base 1408 and to a separate rules base for triggering any required notifications or recommendations. Recommendation engine 1409 may output to Pam's labs data and profile history 1407 and ultimately to Pam's dashboard data visible in Pam's application dashboard available whenever Pam syncs with the website.

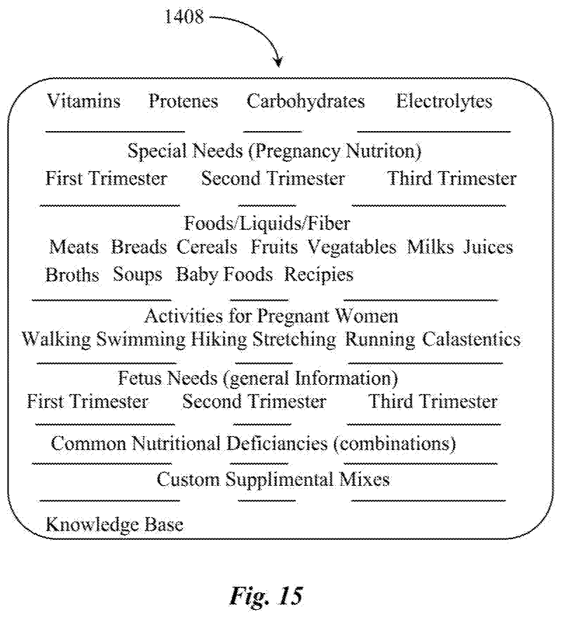

FIG. 15 is a block diagram depicting knowledge base 1408 of FIG. 14. KW base 1408 may include information about vitamins, proteins, carbohydrates, and electrolytes including how they may be absorbed and utilized during the trimesters of pregnancy and after. The knowledge base may include information about special need relative to pregnancy nutrition and may include proprietary mix formulas for nutritional supplements. In general, the GYFT™ (trade name) system includes special vitamin mixes for the first second and third trimester.

Knowledge Base 1408 may include information about foods, liquids and fibers such as meats; breads; cereals; fruits; vegetables; milks; juices; broths; soups; baby foods; and recipes. Knowledge base 1408 may include information relative to activities a pregnant client might engage in for fitness including walking; swimming; hiking; stretching; running; yoga; Pilates; and calisthenics.

Knowledge base 1408 may include information about needs of a fetus during the first trimester the second trimester and the third trimester. The knowledge base may include information about mother and early childhood nutrition and health needs. Knowledge base 1408 may also include information about common nutritional deficiencies such as vitamin D deficiency, which is very common. Knowledge base 1408 may also include proprietary information about engineered vitamin and supplement mixes.

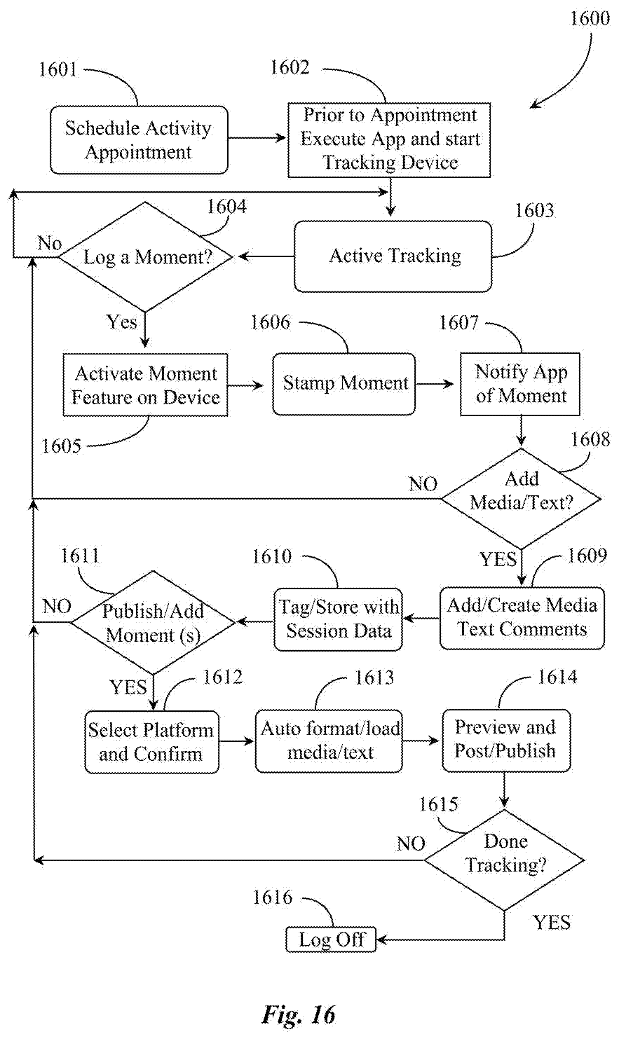

FIG. 16 is a process flow chart depicting steps 1600 for tracking a moment and associating media to the moment according to an embodiment of the present invention. A step 1601 a pregnant client may schedule an activity for tracking. The client may schedule a reminder to track notification in one embodiment. At step 1602, the client prior to the scheduled appointment or activity, may execute the client application and initiate function of the pendant tracking device. One example of an activity may be a scheduled pregnancy ultrasound appointment for checking health and status of the baby. At step 1603, it is presumed that the client is engaging in the activity and the pendant is gathering data and is recognized and paired for communication with the client phone and application. The client application may run in a background mode without displaying or otherwise obstructing any other smart phone functions.

Any time during the activity, the client may determine to record or bookmark a moment in time during the tracked activity at step 1604. A moment is the time frame of an event that may occur during pregnancy such as a kick, an ultrasound session. A moment may also be determined by a client such as a visit from a relative, a touching baby shower gift, a moment of reflection, or any point in time the client decides to record. If the client determines not to bookmark a moment in step 1604 and the activity is ongoing, the process simply resolves back to data gathering. If the client determines to record or bookmark a moment at step 1604, then the client may depress or squeeze the UI moment feature on the pendant for a period such as for two seconds at step 1605.

At step 1606, the pendant creates a date and time stamp defining the moment. The pendant may vibrate to confirm that a moment has been time stamped and associated with appropriate data like location of the client and the subject of the scheduled activity if desired. FW on the pendant device provides instruction for creating and logging a moment. A client may define a moment at any time the pendant tracking device is on whether the activity is scheduled or not and whether the client phone or application is active. In this process it may be assumed that the client's phone is on and that the application is running. At step 1607, the pendant may notify the application over wireless connection that the moment was created.

It should be noted herein that a pregnant client may create and log moments of her pregnancy on the pendant device and just keep them logged on the device until the client executes the client application and pairs with the pendant to access the data wirelessly from the pendant. However, in this aspect, the client application is running, and the smart phone and the pendant are already paired for communication. This aspect brings up a possibility for moment association to existing and or new media and content that may be provided by the client. The pendant may immediately send gathered data and created moments to the smart phone application in real time.

At step 1608, the client may be prompted by the application asking the client whether they wish to add or associate media with the last received moment. Therefore, the client may have an opportunity to take video or pictures, record audio, add text captions or moment descriptions, or even select from system-defined general moment titles that are compiled from logging previous client moments.

If at step 1608, the client does not wish to create or associate content with the moment received by the application, then the process may simply resolve back to data tracking at step 1603. There is no limit to a number of moments a client may track in one tracking session. If the client determines to create or associate media with a moment, the client may do so at step 1609. The smart phone client application may include application program interfaces to third party services that may publish data and may also include its own social interaction platform for friends and family of the pregnant client to visit and view and comment on moment posts that the client may publish. Therefore, at step 1610, the client application may map the most recent media content created by the client and or media content received on the client smart phone to the moment last received from the pendant by the client application.

It is noted herein that it is possible that the client log more than one moment before determining whether to add or associate media with the moments created. In such a case, the client may create or associate media content with each moment logged. Mapping media on the client smart phone to correct moments logged by the pendant enables the client to avoid scrolling through potentially hundreds of photos, videos to select ones that go with the moment. At step 1611, the client application may prompt the client if they wish to publish any moments with tagged media content.

A moment with media may be formatted for post on any one or all of the client's personal social media accounts. In one embodiment, the service provider may have business contracts with one or more online publishing platforms such as Mixbook™, for example. Mixbook™ is a storyboard publication platform for sharing photo albums and family stories and the like for friends and relatives to view. A pregnant client may be offered an account with a contracted publisher like Mixbook™ to publish moments from her pregnancy.

If a client decides not to publish a moment at step 1611, the process may resolve back to active tracking at step 1603. In one aspect, the client may wait to publish moments or may decide not to publish moments. The process is completely client-driven where the service provides mapping and formatting support. If the client desires to publish a moment at step 1611, the client may select a desired project or platform at step 1612 for publishing. In one aspect, the service provides API support for a client controlled social media account including media constraints and formatting for post with client preview. In one aspect the client application may present a list of possible publishing vehicles including through the application to a public posting site where the client may control the viewing audience. In one aspect the client may add moments recorded throughout the client's history of pregnancy to a picture storyboard that may be shared, printed and mailed, etc.

At step 1613, the client application may retrieve the tagged media content associated with the moment being published or added to a storyboard and formats the media content in the template of the publishing vehicle that also offers a client preview and a connect and share button. At step 1614, the client may preview the moment and edit or post or otherwise add to a storyboard. At step 1615, the client may decide whether to discontinue the tracking activity (done tracking). It may be noted herein that discontinuing tracking may end anytime during the overall process and that a client may decide not to publish a moment or moments in real time. The client may post real-time or at a later date or not at all

If the client decides they are not done tracking at step 1615 the process may resolve back to step 1603 for further tracking. It is also noted that the client may track activity with the pendant on but without being connected to the smart phone application. If the client creates moments and eventually transfers them to the client application, then the client application may organize them and present them on an interactive screen that enable the client to select moments for classification wherein opportunity to add media content and text description is provided at that time and after the tracked activity has expired. If the client is finished tracking in step 1615 the client may log off at step 1616 (if connected to website). In one aspect the client may toggle the pendant device from active tracking mode to a sleep mode that saves energy. In a variation of this aspect the sleep mode command may override the pendant function of waking up based on detection of motion. In the same aspect, the sleep mode may be disabled by the client restoring the pendant to previous mode of waking and tracking data after detecting motion like a client picking the device up and donning the device.

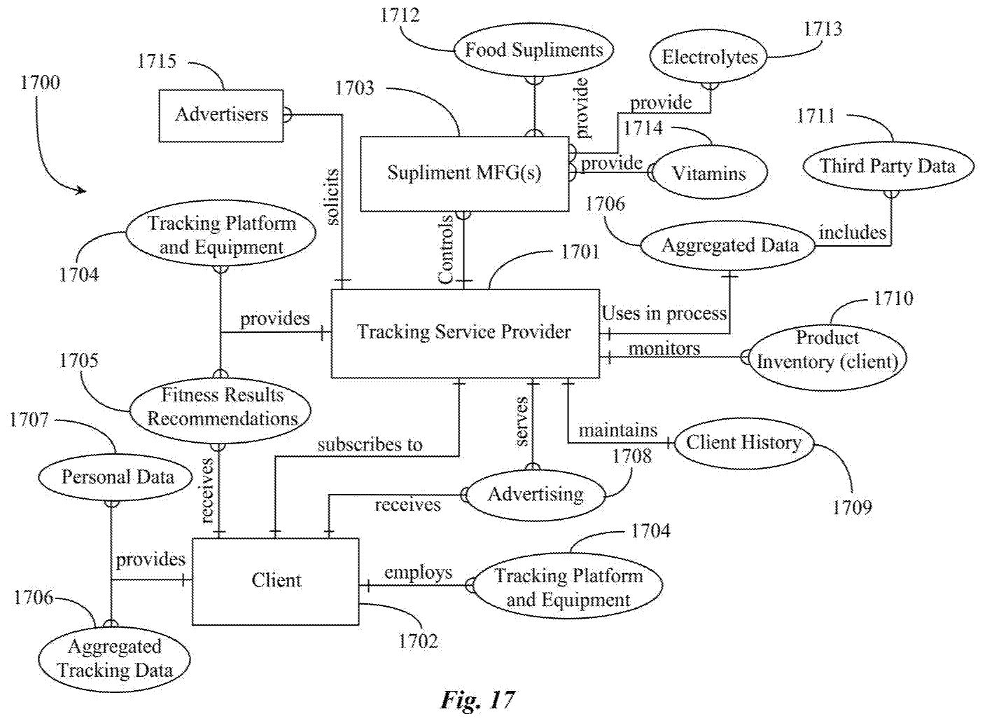

FIG. 17 is a united modeling language diagram depicting a business and service model 1700 representing an abstract business concept model for practicing the invention from a service provider perspective. Model 1700 begins with a tracking service provider 1701. Service provider 1701 may provide service to one or more than one client 1702 who subscribes to the tracking service. The tracking service provider 1701 provides platform (SW) and tracking equipment (connected devices) 1704 and fitness tracking results and recommendations to the client(s) including recommendations to purchase proprietary mixed nutritional supplements including vitamins, electrolytes, and proteins including shakes and bars. The client 1702 employs the tracking platform and tracking equipment to provide aggregated data to the service provider 1701.

As a subscriber, the client receives/has access to tracking platform and equipment 1704 and fitness results and recommendations 1705. The client may subscribe to service provided by the service provider 1701 for the period including three trimesters of pregnancy (about 280 days) and after birth of the baby for approximately 720 days of early childhood. The client may purchase products through the platform anytime during the service period. The client employs the platform and tracking equipment 1704 and the client provides personal data 1707 and aggregated tracking data to the tracking service provider 1701.

Tracking service provider 1701 controls one or more supplement manufacturers (MFG) 1703. MFG(s) 1703 provide mixed food supplements 1712, mixed electrolytes 1713, and mixed vitamins 1714. The strategy for mixing consumable products follows the general knowledge attributed to the general condition of pregnancy and the consumables mentioned above may be mixed differently relative to content and ratio for each trimester period of pregnancy. For example, vitamins may be separated relative to mix and content according to trimester and packaged accordingly for the pregnant client.

In one embodiment, tracking service provider 1701 may also control or contract with at least one baby product manufacturer to provide the pregnant client with vetted baby products after the birth of the baby and into the 720 days of early childhood. One viable product required by all mothers is diapers. The service of the invention provides clients with seamless access to organic diapers, in one case, bamboo diapers that may be made available through the client application. In one embodiment service provider 1701 controls a line of postnatal baby products like organic diapers, organic baby wipes organic lotions, organic powders, and baby foods or formulas.

Tracking service provider 1701 monitors product inventory 1710 on behalf of the client. This is accomplished due to client logging of intake of consumables made available to the client. The client has a regular product use history compiled in real time, so the service may monitor and make adjustments in supply such as for an ongoing purchase order for vitamin supplements or for bamboo diapers, or other mentioned products. Tracking service provider 1701 maintains client history 1709. Client history 1709 may include labs data, purchase history, and product use history. Tracking service provider 1701 uses aggregated data 1706 about the client including aggregated tracking data from one or more than one client device in processing to provide the tracking results (user labs history). Data aggregated also includes third party data 1711.

Tracking service provider 1701 may solicit one or more advertisers 1715 and may server advertising 1708 to clients wherein that advertising is displayed in the client application and may be interacted with by the client. The client 1702 may receive advertisements or advertising 1708. In one aspect of the model, the tracking service provider 1701 may vet certain advertised products and services as being beneficial to the service providers clients 1702 and therefore solicits those advertisers to purchase advertising space in the client applications. For example, a local Pilates class designed for pregnant women may be available to clients of the service in a region of coverage. The tracking provider wishes to make the knowledge of the resource available to the client as part of the service model because it benefits the health of the client during her pregnancy or after her pregnancy as well. Similarly, the service provider may vet other products and services and make determinations whether to make those ads available to the client base.

It is noted herein that there may be attributes in the overall business model not listed in this diagram like client 1702 receiving shipments of products and consumables (vitamins, food supplements, proteins, and electrolytes). Shipments to the client are resulting of client interaction through the client application with the service recommendations to purchase product for consumption and with vetted advertisers to purchase other products. It is noted herein that the tracking service provider may provide proprietary products manufactured under its control and ownership with proprietary packaging designed specifically for the service client base. It is also noted that third party products and services may also be marketed to clients through the client application.

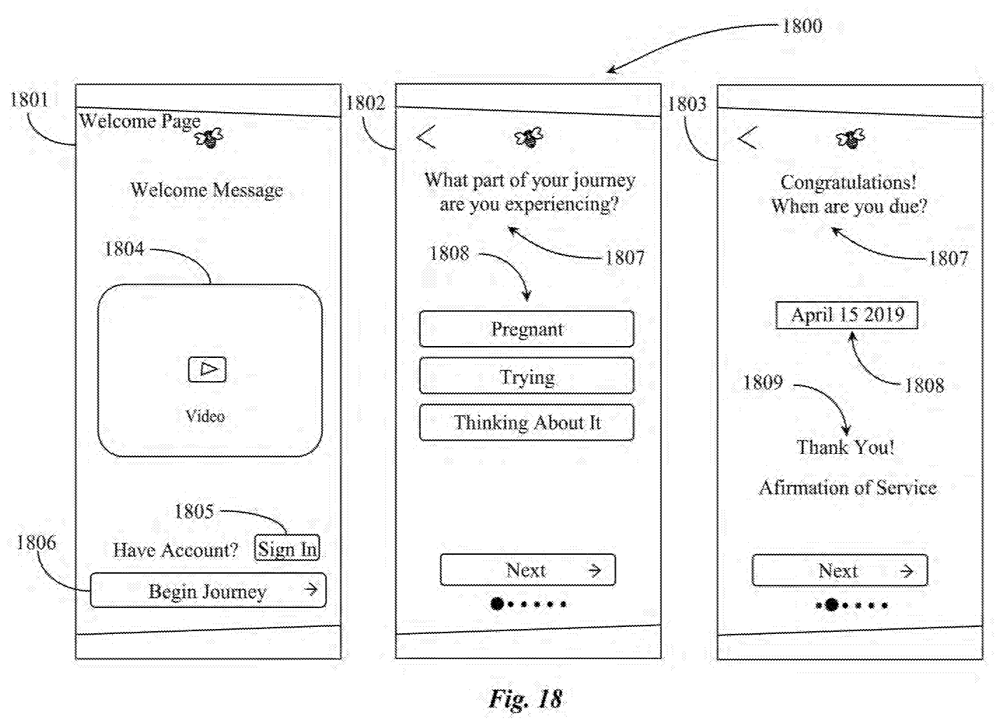

FIG. 18 is a block diagram depicting a set of exemplary application screen shots 1800 of client application 113 of FIG. 1. Screen shots 1800 include a welcome screen or page 1801 that may display for a new client when the first execute their client application on their smart phone device. Screen 1801 is formatted for display on a smart phone analogous to smart phone 109 of FIG. 1. At top of the screen there may be a provider logo and welcome message to the client. Typical display data showing the state of the smart phone can be time, date etc. may be visible in the top pane of the screen.

An informational video 1804 or other media content may be provided for client review to learn more about or become orientated with the service. In one embodiment, a contributor of the screen content may also be a contributor to the knowledge base information put together to enable better optimization of health in general with finer granularity. Screen 1801 may include a prompt to the client asking if the client has an account. A sign in option 1805 may be provided for a client already having an account set up to sign into their account. Otherwise, the client may proceed to create an account through the application by selecting option 1806 (begin journey) and navigating to a next screen.

A next screen 1802 may include a system prompt 1807 asking where in or what part of the journey the client is in followed by interactive options 1808 to answer the question. The context of a subsequent screen may depend upon how the client answered the question on screen 1802. In this case it is presumed the client selected the pregnant option of options 1808 and then selected next to get screen 1803. At the bottom of screen 1802 and in subsequent screens, the application may track your progress through the account set-up process.

A next screen 1803 begins with a system prompt 1807 to the client that congratulates the client on her pregnancy and asks when the baby is due. The system may provide a calendar function 1808 for the client to select the due date, which in this case is Apr. 15, 2019. System interaction sequences with the client are engineered to be friendlier and encouraging to the client rather than clinical language prompts. After the client selects the babies due date the system prompt 1809 may thank the client and confirm affirmation of dedicated service to the client. The client may hit next to navigate to a next screen.

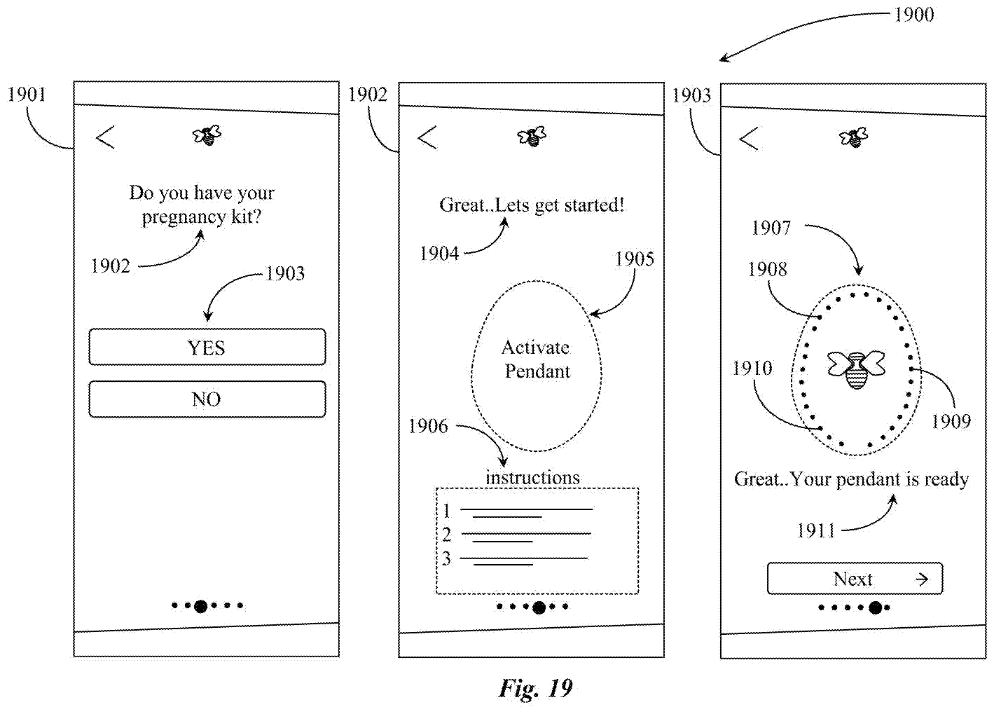

FIG. 19 is a block diagram depicting a set of exemplary application screen shots 1900 of client application 113 of FIG. 1. Screen shots 1900 include a screen 1901 beginning with a system prompt 1902 asking the client if they have purchased their pregnancy starting kit. The pregnancy kit may include at least the pendant and at least the first trimester of pregnancy vitamins. The system prompt 1902 may include a yes or no selection option 1903. In one embodiment, a client may use natural language spoken voice to answer synthetic voice prompts through the application. If the client confirms she does not have the pregnancy kit, the next set of screens may be adapted to carry out that transaction. If the client confirms ownership of the kit, then a next screen 1902 may display with a beginning system prompt 1904 commending the client and suggesting we get started.

Screen 1902 may include a pendant device activation window 1905 and a set of instructions 1906 comprising a three-step method for activating the pendant. For example, a first step may be to charge the pendant device with the charging cradle provided with the device. A second step may be to note the pendant is fully charged when the LED lights stop blinking. And the final step may be to place the pendant on the screen in the activation window, so the service may unlock the device or otherwise activate the tracking device as a personal device of the client.

A next screen 1903 may display a virtual representation 1907 of the pendant tracking device that depicts the three tracked data categories the device displays LED status on, more particularly steps taken category 1910, ounces consumed category 1908 (hydration), and calories burned category 1909. The client application may collect hydration and nutrition data through your voice. If the pregnant client has a smart bottle, the application may sync with the bottle to automatically collect the hydration data.

Each physical category is divided into 10 progressive micro lights that illuminate based on a percentage of a total goal attained such as if 200 calories have been burned of a 1000 calorie goal then two microlights may be lit to depict the status. The pendant minimally tracks motion, direction of motion, the location of the client (GPS), distance traveled, duration of activity, and date time of activity. The pendant may also in some embodiments track elevation through barometric function, track client orientation through triangulation, track heart and or respiratory rate via sensor, and track general UV intensity via sensor. The system may prompt the client with an affirmation of success at 1911 to confirm to the new client that the pendant is ready to track data. The client may select next to navigate to a next screen.

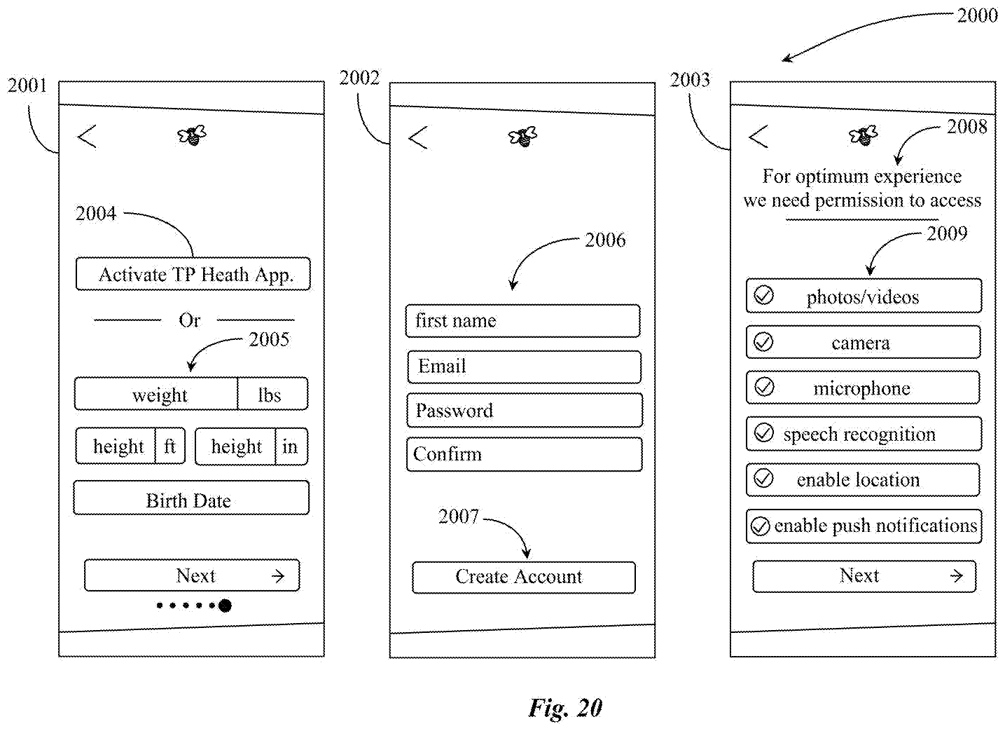

FIG. 20 is a block diagram depicting a set of exemplary application screen shots 2000 of client application 113 of FIG. 1. Screen shots 2000 include a screen 2001 adapted to gather health data about the pregnant client. The system may present an option 2004 for the client to activate a personal third-party health application already containing the client's health data and importing the appropriate data from the application to the client application (may require permission from the client). In one embodiment the client may begin populating an electronic data form 2005 containing the required data fields.

There may be several more screens presented for client population with health-related data. Once that is accomplished, a next screen 2002 may prompt the client to enter authentication data 2006 including name, email address, and a password for authentication at the next log-in. In one embodiment the client may be given a security token for authentication with the back-end servers and for decrypting dashboard data for personal viewing in the client application. The client data may be held in encrypted format at the web service and at the back-end service location. An option 2007 may be provided to create an active account. By selecting option 2007 an account is created for the client.

A subsequent screen 2003 may be displayed the screen beginning with a system prompt 2008 to the client asking for permissions to access and control certain functions on the client smart phone for optimizing client experience. A list 2009 of configurable items may be presented to the client for client review and selection. The items listed include photos/videos, camera/microphone, speech recognition (client app), GPS location services, and push notifications.

GPS location enables the tracking service to determine location and retrieve third party information based on location. Push notifications enable the service to push notifications to the smart phone application and over Bluetooth™ to individual devices like the pendant or the connected water bottle. In one embodiment, the client may select all in the list or some in the list as it is client driven and the client maintains control over their own data. The client may select next and navigate to a next screen.

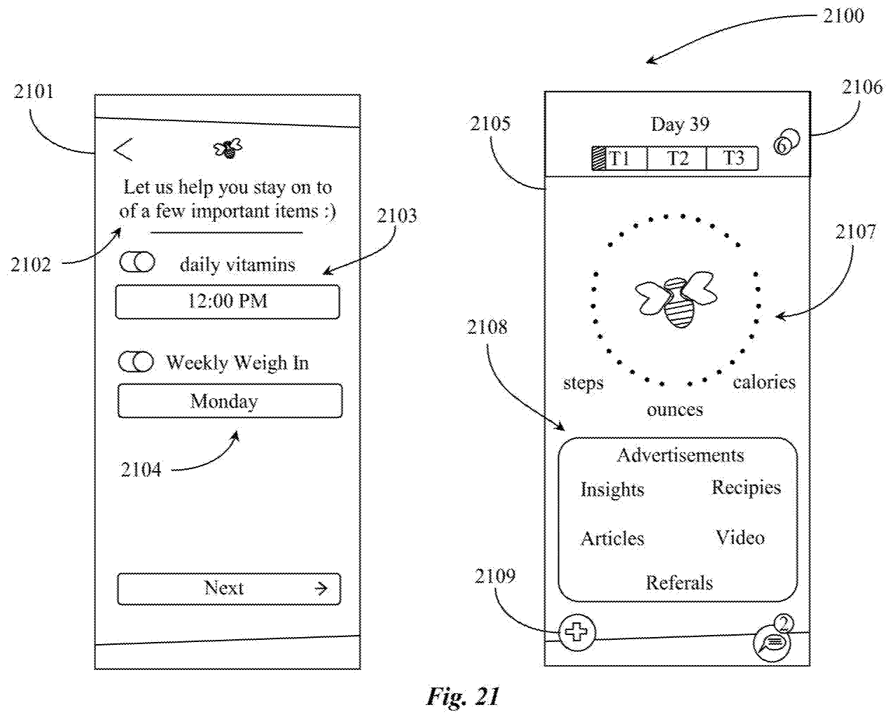

FIG. 21 is a block diagram depicting a set of exemplary application screen shots 2100 of client application 113 of FIG. 1. Screen shots 2100 include a screen 2101 adapted to help the client set up regular reminders or notifications. Screen 2100 begins with a system prompt 2102 to the pregnant client to invite the client to allow the system to set up reminders to perform certain activities. The system may provide a list of activities the client may set up and receive notifications on including an activity 2103 to take daily vitamins and an activity 2104 to weigh in and report weight weekly wherein the client has selected Monday as a weigh in day. In one embodiment, a pregnant client may utilize the smart scale of FIG. 1 to weigh in so that the system may calculate and provide granular results back to the client like total fat, total water content, lean muscle mass, bone mass, bone density, etc. In a variation of this embodiment, the system may also calculate and separate the baby weight from the mother's weight.

The client may select next to navigate to a next screen 2105 adapted to display summary labs data calculated from the client's first use. Screen 2105 may be a dashboard screen and has a header 2106 that indicates to the client, the current progression of the client through the trimesters of her pregnancy. In this example, the client is on day 39 of the first trimester. Client notifications (6 unread notifications) are depicted upper right from the trimester scale.

Screen 2105 includes a status window 2107 provided in the likeness of the client pendant device with the three categories of resulting data display the device itself depicts which are steps taken (motion), ounces consumed (hydration), and calories burned (nutrition). Screen 2105 includes media content 2108 delivered to the client through the application. Media content forwarded or published to the client through the client application may include but shall not be limited to advertisements, insights, recipes, articles, video/audio, and referrals. Unread messages (2) waiting for the client are depicted at the lower right corner of screen 2105.

At the lower left portion of screen 2105, an add content/site mapping option 2109 is provided that enable the client to add content, content categories, order standard views of data, access calendar, to perform or configure other available service options, or to schedule future tracked activities. It is noted herein that the client application analogous to application 113 of FIG. 1 is a browser-based application and there may be other navigation tools available to the client operating from within the application like back and forward options, home page option, data only vs data charts toggle, messaging link, moment publishing link, history link, etc. It is also noted herein that with push notifications set to on, the client receives automatic sync and notification and reminders sent from the service to the client application and certain notifications may be automatically propagated to connected client devices like the pendant device or the connected water bottle.

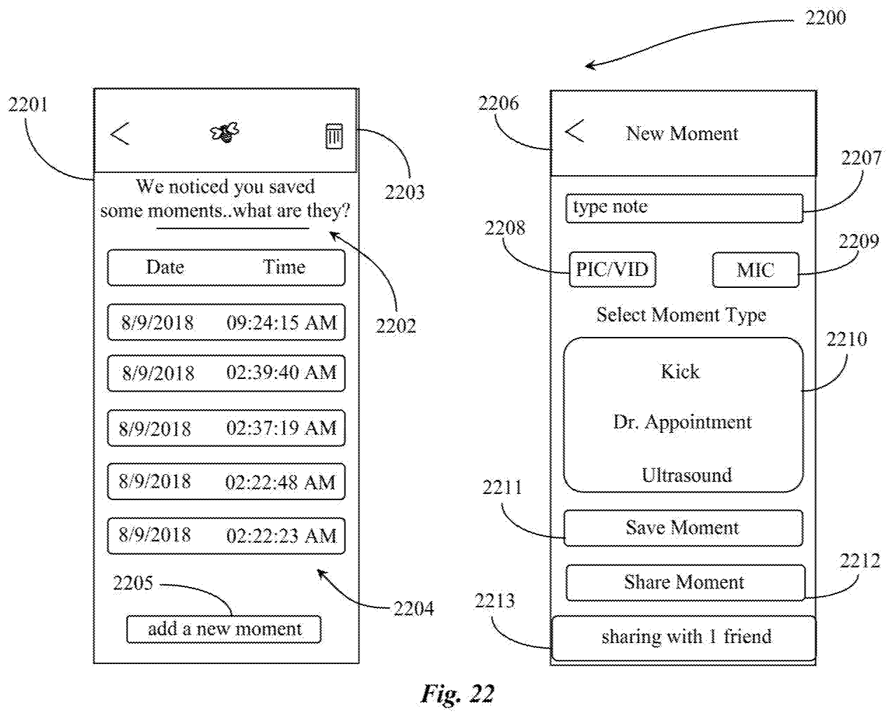

FIG. 22 is a block diagram depicting a set of exemplary application screen shots 2200 of client application 113 of FIG. 1. Screen shots 2200 include a screen 2201 adapted to help a client classify add content to, and share moments saved by the client during one or more tracked activities. In this example, the moments are automatically gathered in the application and displayed to the client when the client syncs with the server.

Screen 2201 begins with a system prompt 2202 to the client reminding the client of the moments saved and asking the client if they wish to go through and classify them. The saved moments may be displayed in an interactive list 2204 where each item is an interactive button enabling further pages and options for classification, adding media, and sharing with friends and family. The moments in list 2204 all have the same date stamp and occurred during a sleep period tracked by the client, the first four moments saved relatively close to one another between 2:22 AM and 2:39 AM may be kicks made by the baby or perhaps feeding and burping of the baby with the last moment saved at 9:24 AM. A trash or delete option 2203 is provided that allows a user to delete any moments the client does not wish to classify.

Screen 2201 includes an interactive option for the client to add a new moment. A moment is saved on the pendant device wherein adding a new moment from the client application initiates connection to the pendant and syncing with the pendant to add the new moment saved on the pendant. In another example, a client may select add new moment 2205 to physically document a fresh moment directly into the application without using the pendant to time stamp the moment, the client application provides date and time stamp if the client adds a new moment not recorded at the pendant device.

Clicking on option 2205 may bring up a next screen 2206 adapted to help the client classify a new moment. A new moment may be any moment in list 2204 that has not yet been classified or a new moment stamped by the client application at the direction of the client. Screen 2206 may include a data form field 2207 for the client to type in a detailed description of the moment. In one embodiment, the system may provide a list 2210 of system-created moment types based on reporting from clients.

Screen 2205 may include option 2208 for adding a photo or a video to the moment and an option 2209 for adding audio recording content to the moment. In one embodiment, the client has already created video or photos of the moment when the moment was taken. In this case the client application may quickly retrieve the media most closely associated with the date and time of the moment saving browse media time for the client. After a moment is classified and media is added, the system may provide an option 2211 to the client for saving the classified moment and associated media.

Screen 2205 includes a system provided option 2212 for the client to share a classified moment. Selecting the option share moment may bring up a list of share options for the client that are possible through the client application. These options may include both programs native to the service such as its own social media site, chat site, or posting site, and popular third-party programs the client may have accounts with such as Facebook™, Instagram™, Snap Chat™. The client SW may include APIs for quickly enabling the client application to group media and format for sharing on a selected program before connecting to the platform server of the selected platform to share.

The client may also preview the moment content as it would appear on the platform selected for sharing and make edits before sharing on the platform. The system may provide a confirmation of a successful publishing of a moment such as sharing with 1 friend in this example. In one embodiment, moments saved may be added to a mother-baby digital story book that may be later printed and distributed to friends and family members. The client may also have access to publishing tools for creating birthday cards, Christmas cards, Calendars, invitations, etc.

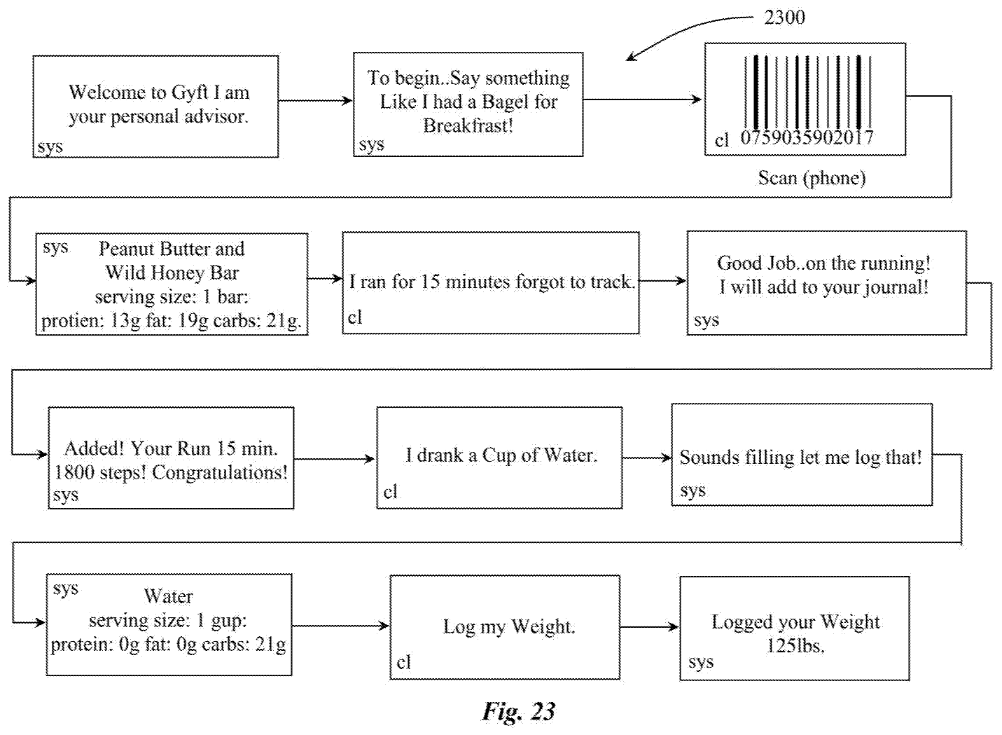

FIG. 23 is a block diagram depicting an artificial intelligence (AI) interaction sequence 2300 between a system personal coaching application and a pregnant client. Sequence 2300 may take place within the client application AI chat interface where the sequence plays out on subsequent chat windows or client application screens. The interaction is depicted in interactive voice response (IVR) sequence format for discussion purposes. Sequence 2300 may occur anytime the mode is selected, and the client may speak in a natural language voice to populate the chat responses to the virtual adviser. The system may use voice recognition to understand the client and may assemble direct responses to the client using text back to the client or synthesized voice back to the client.

In this example, the system adviser begins by welcoming the client to the GYFT™ system, a trade name for the service model, and introducing to the client as a personal adviser. The intelligence accessible to the virtual assistant is the wealth of knowledge in the knowledge base. The system may coach the client to begin speaking or say something to start an interaction sequence. The system may give an example like “I ate a bagel” to the client as an example of something to say to begin the interaction. In one embodiment the client may respond with a spoken question or statement.

In this case, the client holds a bar code from a protein bar up the client ate to the smart phone to scan it in for the client application. The application may send the code to the back-end service to read the bar code to translate the coded description of the bar and give the client the particulars while adding them to the client's labs data. In the next action the client voices that she ran 15 minutes but forgot to track it. The system may respond with encouragement good job on the running! But ignore the “forgot to track” part as a sad thing using some comedy or levity in the interaction for client encouragement and promising to add the data to the client journal. A client journal may provide a history of client running for example. The data may also be vetted and added to the client labs data. In such a case, the system may also ask for other information that would have been available if the run were tracked like location of the run, the date and time of the run, the run lasted 15 minutes so the system may access third party data to help calculate the steps and calories burned the client may provide the number of pounces of liquid consumed during the run and just after and any food taken and the client may simply respond in natural language voice.

The system may confirm to the client that the data was calculated and added to the client journal and the client may announce that she just drank a cup of water. The system may respond with some levity, sarcasm, or comedy in a response like “Oh that meal sounds filling let me log that for you”. The system may then show the data that was logged on behalf of the client. The client may then change subject and request a logging of weight.

The client may use the smart scale to log weight without speaking the result into the client application adviser chat sequence. In this case the client may step on a regular scale and say the weight total to the client application for logging, in this case 125 pounds. If the system does not understand a client's utterance, the system may ask the client to repeat that. In one embodiment the client may train online with the voice application to improve voice recognition of the client. A client may log by natural voice a fully complex dinner.

A client may say I just had two bean burritos and pickle ice cream for dinner last night and a 12-ounce bag of Doritos for breakfast. The service may log the time of the meal, the type and description of the meal both brand name or non-brand meals. The system may log based on data about quantity consumed so if a client says hamburger the system may assume quarter pound whereas if the client says double cheese burger, the system knows half pound of beef plus two layers of cheese. The system may categorize all the logged foods and calculate for macro nutrients and micro nutrients and add those to baseline. Multiple languages are supported.

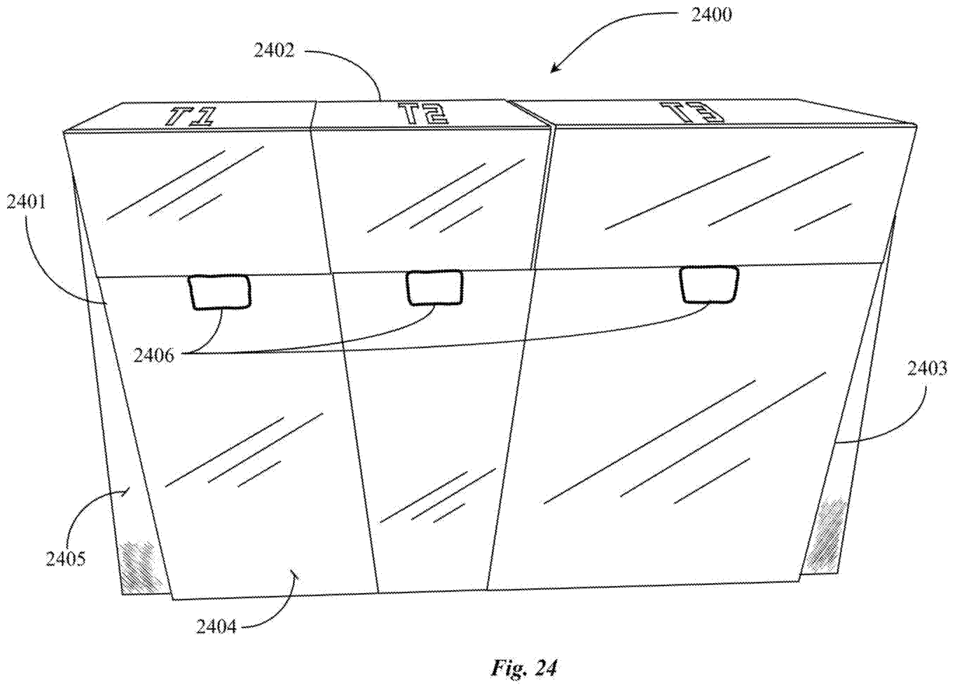

FIG. 24 is a perspective view of a pregnancy kit package 2400 adapted to hold specially mixed vitamins for a client, the vitamins separated in the packaging by trimester that they are mixed for. In this case the service provider provides engineered vitamins in pill form or in another form such as a liquid. Blister packs containing engineered pills may be provided or liquid sachets containing liquid vitamin supplements may be provided. Each may contain one day's worth vitamins selected and mixed according to which trimester they are engineered for. So, a client would naturally start with the first packet of the first trimester then daily until all the vitamins have been taken.

In one embodiment, these daily events (taking a days' worth of vitamins) are logged in client labs history and levels attributed to baseline or goal-oriented health are continually reviewed in light of nutritional intake including vitamins. Recommendations may also be made such as if nutritional intake from another source obfuscates the requirement to dose with vitamins for one day. The service of the invention takes into account some 30 micro nutrients in about 200 million foods. Clinical trials whose results are available to the inventor have been conducted to help refine the mixing strategy of the daily vitamins for pregnancy relative to the separate trimesters of pregnancy.

Packaging 2400 includes three separate package compartments labeled for the correct trimester T1 (compartment 2401), T2, (compartment 2402 and T3 (compartment 2403). Each compartment fits into an adjacent compartment by design of an outer skin and an underlying skin representing the packaging walls. For example, surface 2404 represents the outer packaging skin including box openings fashioned of the outer skin 2404 that may be magnetized on the inner wall surface to stick to the underlying skin 2405.

Embedded magnets may be provided in both materials to promote the materials adhering to one another. Package 2400 may be pulled apart into three separate packages by breaking the magnetic force holding the compartments together. Likewise, the opening flaps are also magnetized to stick and to stay down over the compartment openings. Each compartment holds all the vitamins needed to fill out the trimester. Blister packs may be provided for dry pills and liquids may come in liquid sachets. Each serving size in one day's worth of the vitamins.

It will be apparent to one with skill in the art that the personal fitness tracking and recommendation system of the invention may be provided using some or all the mentioned features and components without departing from the spirit and scope of the present invention. It will also be apparent to the skilled artisan that the embodiments described above are specific examples of a single broader invention that may have greater scope than any of the singular descriptions taught. There may be many alterations made in the descriptions without departing from the spirit and scope of the present invention.

It will be apparent to the skilled person that the arrangement of elements and functionality for the invention is described in different embodiments in which each is exemplary of an implementation of the invention. These exemplary descriptions do not preclude other implementations and use cases not described in detail. The elements and functions may vary, as there are a variety of ways the hardware may be implemented and in which the software may be provided within the scope of the invention. The invention is limited only by the breadth of the claims below.

1. An executable network-hosted service model comprising:
at least one server controlled by a service provider entity, the at least one server connected to the network and to at least one data repository;
software executed by the server from a non-transitory computer readable medium coupled to or otherwise accessible to the server, the software including at least one set of machine readable instructions, the instructions causing the server to perform the following tasks;
(a) receiving over the network from a service beneficiary having a current medical condition, activity data recorded by at least one dedicated activity data tracking device controlled by the service beneficiary, the at least one data tracking device adapted for at least short-range wireless communication with at least one other electronics device;
(b) identifying the service beneficiary, locating data previously stored about the service beneficiary and general knowledge data stored about the current medical condition of the service beneficiary, and mapping the activity data received in (a) to the located beneficiary data and knowledge data;
(c) retrieving available third party held data from one or more other servers connected to the network, the third-party data requested having at least some relevancy to the recorded activity data;
(d) processing the activity data for calculable data results, the calculation performed considering the information stored about the service beneficiary and considering the third-party data retrieved in (c);
(e) updating the data stored about the service beneficiary in the at least one data repository with the new data results calculated in (d);
(f) requesting through at least one generated recommendation notification sent to the service beneficiary over the network at least one transaction item to be initiated by the service beneficiary and fulfilled by the service provider; and
(g) completing at least one transaction item initiated by the service beneficiary in (f), including fulfilling the order of the transaction.

2. The executable network-hosted service model of claim 1, wherein the at least one dedicated activity tracking device includes a wearable electronic pendant including a sensor tracking at least motion, time of tracked motion, and direction and distance of tracked motion.

3. The executable network-hosted service model of claim 1, wherein the service beneficiary is a woman and the current medical condition is a pregnancy, the service model adapted to determine and address at least the nutritional needs of the service beneficiary through a pregnancy of approximately 280 days and during a post pregnancy period of approximately 720 days.

4. The executable network-hosted service model of claim 3, wherein the data stored about the service beneficiary includes a labs history documenting nutritional intake and baseline health data compiled through repetitive data tracking, result processing, and appendage to the stored data during the service period of pregnancy plus 720 days of early childhood development of the child.

5. The executable network-hosted service model of claim 2, wherein the at least one dedicated data tracking device includes a dedicated electronic liquid containment bottle including a sensor for tracking the amount of liquid the service beneficiary drinks over time.

6. The executable network-hosted service model of claim 5, wherein the service beneficiary employs both the pendant tracking device and the liquid consumption tracking device for a same period of activity tracking.

7. The executable network-hosted service model of claim 1, wherein the activity tracking device communicates tracked data wirelessly to a smart phone controlled by the service beneficiary, wherein the service beneficiary uses the smart phone to navigate online and transfer the tracked activity data to the at least one server over the network the transfer aided by a thin client smart phone application providing connectivity between the service beneficiary and the service provider.

8. The executable network-based service model of claim 5, wherein the at least one dedicated data tracking device includes a dedicated electronic weigh scale including a bio electric impedance program and apparatus for dividing total weight into sub-categories of total weight including at least lean muscle mass, bone mass, and visceral fat percentage.

9. The executable network-hosted service model of claim 7, wherein the activity data recorded by the at least one dedicated tracking device in (a) includes at least one date and time-stamped file created by the service beneficiary as a bookmarked moment created sometime during activity tracking by physically manipulating a user interface indicium provided on the activity tracker.

10. The executable network-based service model of claim 1, wherein the third party held data retrieved in (c) is weather data including actual and forecast data, location data including global position and sea level-based elevation data.

11. The executable network-based service model of claim 10, wherein the weather data and forecast data further include humidity and air quality data.

12. The executable network-based service model of claim 9, wherein the service beneficiary receives notification of one or more moments bookmarked by the service beneficiary and may associate one or more media files to each bookmarked moment and wherein the service beneficiary may provide authorization to the service provider to publish the one or more characterized moments to a list of friends and family made available to the service provider by the service beneficiary.

13. The executable network-based service model of claim 1, wherein in (f), the order of the transaction is for vitamins, protein supplements, electrolytes, or a combination thereof for one or more of three trimesters of pregnancy or one or a combination thereof for a postnatal period.

14. The executable network-based service model of claim 13, wherein is (f), the order of the transaction is for one or a combination of organic diapers, organic baby wipes, organic lotions, organic powders, and baby foods or formulas.

15. The executable network-based service model of claim 7, wherein the service beneficiary uses the smart phone aided by the thin client application to view logged data results and to receive notifications of service adjustments to logged data, reminders to take vitamins, reminders to weigh in and report weight data, and notifications requesting authorization to make adjustments to purchase orders with more or less product or to initiate a transaction for a new product.

16. The executable network-based service model of claim 3, wherein the vitamins and the nutrients recommended for the service beneficiary are strategically mixed for each of the three trimesters of pregnancy and for the postnatal period of the service based on a combination of knowledge solicited or contributed and normalized baseline nutritional data logged for the service beneficiary.

17. The executable network-based service model of claim 2 wherein the wearable electronic pendant further includes a sensor for tracking exposure to sunlight, and micro LED powered readouts displaying percentage of or total steps taken, percentage of or total ounces of liquids consumed, and percentage of or total calories burned.

18. The executable network-based service model of claim 2, wherein the wearable electronic pendant may further be worn as a wrist device, or a belt clipped device by installing the pendant into the wearable forms.

19. The executable network-based service model of claim 1, wherein the general knowledge data stored about the current medical condition of the service beneficiary includes at least knowledge data about nutritional deficiencies that may be caused by the medical condition, knowledge data about metabolic shifts that may occur during the medical condition, and knowledge data about hydration level shifts that may occur during the medical condition.

20. The executable network-based service model of claim 1, wherein the network is the Internet network including any connected sub networks.
